(19) United States
(12) Patent Application Publication
(10) Pub. No.: US 2009/0087425 A1
(43) Pub. Date: Apr. 2, 2009

(54) Combination of nitroderivatized steroid and bronchodilator for treating respiratory disease

(75) Inventors: Paolo Renzi, Westmount (CA); Luc Paquet, Sherbrooke (CA); Nicolay Ferrari, Boucherville (CA)

Correspondence Address: Davidson, Davidson & Kappel, LLC, 485 7th Avenue, 14th Floor, New York, NY, 10018, US

(73) Assignee: Topigen Pharmaceuticals Inc., Montreal (CA)

(21) Appl. No.: 12/228,044

(22) Filed: Aug. 8, 2008

Related U.S. Application Data
(60) Provisional application No. 60/955,168, filed on Aug. 10, 2007.
(60) Provisional application No. 61/065,401, filed on Feb. 8, 2008.
(60) Provisional application No. 61/065,400, filed on Feb. 8, 2008.
(60) Provisional application No. 61/065,397, filed on Feb. 8, 2008.

(51) Int. Cl. A61K 31/417; A61K 31/22; A61K 31/58; A61K 31/19; A61K 39/395; A61P 11/00; A61K 31/439

(52) U.S. Cl. 424/130.1; 514/400; 514/546; 514/171; 514/557; 514/291

(57) ABSTRACT

There is provided a pharmaceutical composition for treating respiratory disease comprising an NO-donating steroid and at least one bronchodilator. Also provided is a method for treating respiratory diseases associated with inflammation comprising administering an NO-donating steroid with at least one bronchodilator. Method of use of the combination of an NO-donating compound, and a steroid with at least one bronchodilator for treating respiratory disease is also provided.

### CROSS-REFERENCE TO RELATED APPLICATIONS

This is a continuation-in-part of U.S. Provisional Patent Application Ser. No. 60/955,168 filed Aug. 10, 2007; Ser. No. 61/065,397, filed Feb. 8, 2008; Ser. No. 61/065,400, filed Feb. 8, 2008; and Ser. No. 61/065,401, filed Feb. 8, 2008; each of which are hereby incorporated by reference in their entireties.

### FIELD OF THE INVENTION

The present invention relates to compositions comprising NO-donating compounds, such as NO-modified steroids or NO-modified corticosteroids, having improved pharmacological activities, in combination with bronchodilators. The present invention further relates to methods of use of this combination as administered to patients in order to treat respiratory illnesses.

### BACKGROUND OF THE INVENTION

Administration of long-acting inhaled corticosteroids (ICS) in conjunction with long-acting beta-agonists (LABA) has been available for years for the treatment of asthma and chronic obstructive pulmonary diseases, commonly abbreviated as COPD, examples of such being emphysema and chronic bronchitis. For example, the combination of budesonide (an ICS) and formoterol (a LABA) is available under the brand name Symbicort™ and is recommended by the National Asthma Education and Prevention Program of the National Institute of Health for long-term control and prevention of symptoms of moderate and severe persistent asthma. The combination is offered in a dry powder inhaler device marketed as Turbuhaler™ by AstraZeneca. Another combination scheme widely used for the treatment of COPD is a short-acting anticholinergic, ipratropium, plus a short-acting beta-agonist (SABA), albuterol (under the brand name Combivent™).

Nitroderivatized steroids have been widely described in various issued patents as well as patent applications, for example, U.S. Pat. No. 6,610,676 (WO 98/15568) as well U.S. Patent Application Publication Nos. 2006/0052594 (WO 03/064443) and 2007/0238882 (WO 02/094758), the contents of which are hereby incorporated by reference.

One such nitroderivatized steroid compound (designated herein as TPI-1020) is an inhaled NO-donating derivative of budesonide having the following chemical structure (Formula 2):

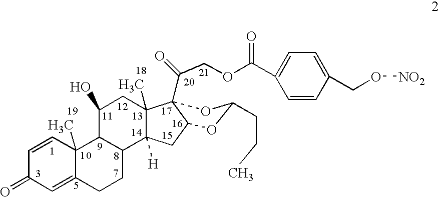

Budesonide is a widely prescribed, multi-billion dollar ICS, marketed worldwide by AstraZeneca under the name Pulmicort®. In pre-clinical studies, TPI-1020 was shown to have improved and broader pharmacotherapeutic properties over budesonide. TPI-1020 was shown to significantly block the recruitment of inflammatory cells (neutrophils) in rodent models of airway inflammation and pulmonary disease. Neutrophils are immune cells that play a central role in the pathology and exacerbations of COPD. TPI-1020 was also shown to significantly reduce release in the lung of key mediators of inflammation.

Salbutamol is a short-acting beta-agonist (SABA) that directly stimulates the lungs to open by binding to the beta-receptor sites on smooth muscles. Salbutamol is used to treat bronchospasm (wheezing, coughing and shortness of breath) associated with pulmonary obstructive problems such as asthma, bronchitis, emphysema and other lung-related problems such as Chronic Obstructive Pulmonary Disease (COPD). Salbutamol and other beta-agonists work by relaxing the muscles of air passages in the lungs so that air flow can circulate into them more freely and improve breathing.

Formoterol is a long-acting beta-agonist (LABA) that is used as a rescue medication in Europe. However, the only FDA-approved indication in the US is as a preventive LABA. Formoterol has a rapid onset of bronchodilator action and this is mediated mainly by a relaxing effect on contracted airway smooth muscle. Formoterol as a reliever gives rapid and sustained relief of symptoms during an exacerbation. The bronchodilator response to formoterol shows a small reduction during initiation of treatment due to the rapid development of tolerance but this does not progress.

Tiotropium is a long-acting anticholinergic (LAAC) bronchodilator used in the management of COPD. Tiotropium bromide capsules for inhalation are co-marketed by Boehringer-Ingelheim and Pfizer under the trade name Spiriva™. Capsules are inhaled via a proprietary HandiHaler™ device. Tiotropium is a muscarinic receptor antagonist, often referred to as an antimuscarinic or anticholinergic agent. Although it does not display selectivity for specific muscarinic receptors, on topical application it acts mainly on M₃ muscarinic receptors located in the airways to produce smooth muscle relaxation, thus producing a bronchodilating effect.

Chronic obstructive pulmonary disease (COPD) is the fourth leading cause of death in the United States. In 2002 alone, COPD claimed over 120,000 lives. More than 10.7 million adult Americans are thought to have COPD, and approximately 24 million others are thought to have impaired lung function, which is a possible early sign of COPD. Women are more than twice as likely to develop COPD as men, and in 2002, more women than men died due to COPD (61,000 vs. 59,000). The medical costs associated with COPD can be astronomic. In 2004, COPD cost the U.S. health care system an estimated $37.2 billion, including direct medical costs of $20.9 billion, $7.4 billion in indirect morbidity, and $8.9 billion in indirect mortality costs. Although no available drugs halt the long-term decline of lung function associated with COPD, bronchodilators reduce the frequency and severity of exacerbations, improve health status, and improve exercise tolerance. As a class, bronchodilators improve forced expiratory volume in 1 second (FEV1) and some other spirometric parameters by reducing smooth muscle tone. It is important to note that some patients may experience improvements in exercise tolerance without a corresponding improvement in FEV1. Bronchodilators can also reduce air trapping and hyperinflation, two of the key pathophysiologic abnormalities seen in patients with COPD, at rest and during physical activity.

Asthma is an inflammatory disease of the lower airways characterized by reversible airway obstruction and bronchial hyper-responsiveness. It is one of the most common diseases in industrialized countries affecting approximately 130 million people globally. The USA is the fastest growing asthma market (17% growth per annum projected for 2001-2006 versus 5% for Europe) and it is also the largest (48% of the total asthma market versus 33% for Europe). There were 17 million diagnosed asthma sufferers in the USA in 2001.

Other examples of respiratory diseases where inflammation may play a role include: eosinophilic cough, bronchitis, sarcoidosis, pulmonary fibrosis, pulmonary hypertension, cystic fibrosis, and bronchiectasis. Asthma is defined by airway inflammation, reversible obstruction and airway hyperresponsiveness. In this disease the inflammatory cells that are involved are predominantly eosinophils, T lymphocytes and mast cells, although neutrophils and macrophages may also be important. Vast arrays of cytokines and chemokines have been shown to be increased in the airways and play a role in the pathophysiology of this disease by promoting inflammation, obstruction and hyperresponsiveness.

Acute bronchitis is an acute disease that occurs during an infection or irritating event for example by pollution, dust, gas or chemicals, of the lower airways. Chronic bronchitis is defined by the presence of cough and phlegm production on most days for at least 3 months of the year, for 2 years. One can also find during acute or chronic bronchitis within the airways inflammatory cells, mostly neutrophils, with a broad array of chemokines and cytokines. These mediators are thought to play a role in the inflammation, symptoms and mucus production that occur with these diseases.

Eosinophilic cough is characterized by chronic cough and the presence of inflammatory cells, mostly eosinophils, within the airways of patients in the absence of airway obstruction or hyperresponsiveness. Several cytokines and chemokines are increased in this disease, although they are mostly eosinophil directed. Eosinophils are recruited and activated within the airways and potentially release enzymes and oxygen radicals that play a role in the perpetuation of inflammation and cough.

Sarcoidosis is a disease of unknown cause where chronic non-caseating granulomas occur within tissue. The lung is the organ most commonly affected. Lung bronchoalveolar lavage shows an increase in lymphocytes, macrophages and sometimes neutrophils and eosinophils. These cells are also recruited and activated by cytokines or chemokines and may be involved in the pathogenesis of the disease.

Pulmonary fibrosis is a disease of lung tissue characterized by progressive and chronic fibrosis (scarring) that will lead to chronic respiratory insufficiency. Different types and causes of pulmonary fibrosis exist but all are characterized by inflammatory cell influx and persistence, activation and proliferation of fibroblasts with collagen deposition in lung tissue. These events seem related to the release of cytokines and chemokines within lung tissue.

### SUMMARY OF THE INVENTION

The subject invention concerns treating a respiratory disease associated with inflammation in human patients comprising the administration to a patient in need thereof, a therapeutically effective amount of:

at least one corticosteroid useful in the treatment of a respiratory disease; and

at least one bronchodilator; and

a NO-donor, wherein the NO-donor is selected from the group consisting of an inactive carrier which can donate a NO— group, a linking group linking the NO-donating moiety to the corticosteroid, and a linking group linking the NO-donating moiety to the bronchodilator. The chemistry relating to linking of the NO-donor to the corticosteroid or bronchodilating agent is well within the skill of the ordinarily skilled artisan using the teachings and knowledge currently available in the art.

Thus, the NO-donor can be linked to the corticosteroid, to the bronchodilator, or is a compound provided separate from the corticosteroid or bronchodilator, i.e., is not chemically linked to the corticosteroid or the bronchodilator. The corticosteroid, bronchodilator, and NO-donor components of the treatment can be administered separately, as separate compositions at different times, but are preferably administered simultaneously or concomitantly. Additionally, the NO-donor can be linked to the corticosteroid component or the bronchodilating component such that a NO-donor linked to a corticosteroid can be administered in combination with a bronchodilator, or a NO-donor linked to a bronchodilator can be administered in combination with a corticosteroid. These agents can be formulated as three separate compositions or can be formulated as combinations, as described into one or more combination compositions.

In one preferred embodiment, the invention provides a pharmaceutical composition comprising a therapeutically effective amount of (i) at least one bronchodilator, or a physiologically acceptable salt or solvate thereof, and (ii) at least one nitroderivatized steroid, preferably a nitroderivatized corticosteroid, or a physiologically acceptable salt or solvate thereof, for treating respiratory disease associated with inflammation in a patient in need thereof.

Preferably, the bronchodilator is selected from the group consisting of a beta-agonist, an anticholinergic, a methylxanthine, and a phosphodiesterase inhibitor, or a physiologically acceptable salt or solvate thereof. In a preferred embodiment, the bronchodilator is provided in an amount suitable to provide an additional therapeutic effect and, more preferably, can be provided wherein the dose of bronchodilator would be suboptimal if administered alone.

A preferred steroidal compound useful in accordance with the subject invention includes a corticosteroid, and more preferably, an inhaled corticosteroid (ICS) as is well known in the art. The steroidal component or corticosteroid is provided in an amount suitable for treatment of a respiratory disease via inhalation in humans and, more preferably, can be provided wherein the dose of corticosteroid would be suboptimal if administered alone.

In a more preferred embodiment, the combination of corticosteroid, bronchodilator, and NO-donor provide a synergistic effect in a human patient as compared to treatment of the patient without one of the agents.

In another preferred embodiment, the composition of the subject invention, or its use, comprises at least one of said corticosteroid, bronchodilator, or NO-donor which are provided at a dose which is suboptimal if administered alone, and provides a significant reduction in bronchoconstriction or airway hyperreaction (AHR) following exposure to a bronchoconstriction-inducing agent, such as histamine or methacholine as compared to a composition substantially free of at least one of said corticosteroid, active agent, or NO-donor. More preferably, the subject composition provides level of reduction in bronchoconstriction that is statistically significant to p<0.05, and most preferably which is statistically significant to p<0.01.

In another aspect of a preferred embodiment of the subject invention, the composition or its use provides a reduction in bronchoconstriction or airway hyperreaction (AHR) of at least 50%, following exposure to a bronchoconstriction-inducing agent, as compared to a composition free of at least one of said corticosteroid, active agent, or NO-donor. More preferably, the reduction of bronchoconstriction or airway hyperreaction (AHR) is between about 70% and about 100%. In a most preferred embodiment, the composition of the subject invention or its use comprises at least one of said corticosteroid, bronchodilator, or NO-donor at a dose which is suboptimal if administered alone.

According to one aspect, the invention provides a novel combination therapy wherein a bronchodilator and a nitroderivatized steroid are administered simultaneously or sequentially to treat respiratory disease in patients, such as relieving bronchoconstriction in patients suffering from chronic pulmonary disease such as asthma and COPD. These nitroderivatized steroid compounds encompass pharmaceutically active compounds having a steroid backbone or residue, preferably a corticosteroid backbone or residue, which are modified to include a NO-donor moiety linked to the steroid or corticosteroid residue at C-11, C-17, or C-21. For convenience of reference, such compounds of the subject invention may be referred to herein as “NO-modified steroids” or “NO-modified corticosteroids.”

In one embodiment, the nitroderivatized steroid is a compound of the following Formula 3:

A-W 3

wherein A is a corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide; and

wherein W is any nitric oxide (“NO”) donating moiety attached, directly or indirectly thereto capable of donating, releasing and/or directly or indirectly transferring any of the three redox forms of nitrogen monoxide (NO⁺, NO⁻, NO.). In a preferred embodiment, W includes a NO-donating moiety that is linked to the steroid backbone by a linking structure that is optionally cleavable in vivo by hydrolase mediated hydrolysis, to patients having a respiratory disease and having viral susceptibility or a viral infection.

A preferred embodiment of the present invention is directed to a composition and method for treating an animal host or patient comprising administering a bronchodilator in combination with a therapeutically effective amount of a compound A-W, above, wherein W is represented by Formula 1, as shown below:

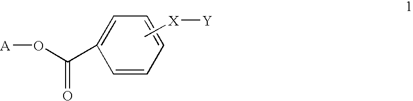

wherein A is the steroid or corticosteroid residue as described above,

X is a C₁-C₅ branched or linear chain alkyl; and

Y is either (ONO₂) or (ONO); with the proviso that A is linked to Formula 1B, directly or indirectly, the C-11 or the C-17 position of the steroid or corticosteroid residue, and preferably at the C-21 position of the steroid or corticosteroid residue when C-21 is present.

According to one aspect, the invention concerns a novel composition comprising a bronchodilator and a nitroderivatized steroid, preferably a nitroderivatized corticosteroid, of the following Formula 4:

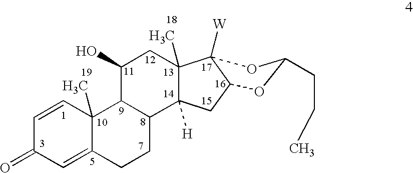

wherein W is any nitric oxide (“NO”) donating moiety attached thereto capable of donating, releasing and/or directly or indirectly transferring any of the three redox forms of nitrogen monoxide (NO⁺, NO⁻, NO.) for the treatment of respiratory disease associated with inflammation in a patient in need thereof. In certain preferred embodiments, W is a moiety as defined herein and is selected from one of the Formulae A-G described in the detailed description portion of this document as well as the appended claims.

In another preferred aspect, the invention provides a method for treating respiratory disease associated with inflammation in a patient in need thereof, comprising the simultaneous or sequential administration of a therapeutically effective amount of

at least one corticosteroid useful in the treatment of a respiratory disease; and

at least one bronchodilator; and

an NO-donor wherein the donor is selected from the group consisting of an inactive carrier, a linking group linking the NO— to the corticosteroid, and a linking group linking the NO— to the bronchodilator.

Preferably, the method of the subject invention comprises administration of a therapeutically effective amount of (i) at least one bronchodilator, or a physiologically acceptable salt or solvate thereof, and (ii) the nitroderivatized steroid of the present invention.

In yet another aspect, the invention provides for methods of use for one or more compsoitons comprising a therapeutically effective amount of (i) at least one bronchodilator, or a physiologically acceptable salt or solvate thereof, and (ii) the nitroderivatized steroid of the present invention or solvate thereof, for treating respiratory disease associated with inflammation in a patient in need thereof.

In still another aspect, the invention provides for the use of a therapeutically effective amount of (i) at least one bronchodilator, or a physiologically acceptable salt or solvate thereof, and (ii) the nitroderivatized steroid of the present invention or solvate thereof in the manufacture of a medicament for treating respiratory disease associated with inflammation in a patient in need thereof.

In another embodiment, the nitroderivatized steroid is administered via inhalation at a dose of from about 100 ug to about 3000 ug. In further embodiments, the nitroderivatized steroid is administered via inhalation at a dose of from about 100 ug to about 2400 ug, from about 200 ug to about 2000 ug, from about 400 ug to about 1500 ug, or from about 600 ug to about 1200 ug.

A most preferred embodiment of the invention comprises administering a bronchodilator in combination with budesonide 21-(4′-nitrooxymethyl)benzoate (designated as “TPI-1020”) or a composition comprising TPI-1020.

In one aspect, the respiratory disease is a chronic pulmonary disease. In another aspect, the chronic pulmonary disease is selected from asthma and COPD.

Other objects, features and advantages of the present invention will be apparent to those of ordinary skill in the art in view of the following detailed description of the invention and accompanying drawings.

### BRIEF DESCRIPTION OF THE DRAWINGS

The present invention will now be described in greater detail having regard to the appended drawings in which:

FIGS. 1A through 1D show the responsiveness of the airways to treatment on bronchoconstriction induced by histamine, as measured by specific airway conductance (sG_{aw}). The results show % sG_{aw} for animals treated with either the vehicle, or: various doses of TPI-1020 (FIG. 1A), 0.44 mg/ml budesonide (FIG. 1B), various doses of salbutamol (FIG. 1C) or a combination of various doses of TPI-1020 and a constant dose of salbutamol (FIG. 1D).

FIGS. 2A through 2D show another set of experiments for determining responsiveness of the airways to treatment on bronchoconstriction induced by histamine, as measured by specific airway conductance (sG_{aw}) The results show % sG_{aw} for animals treated with either the vehicle or: various doses of TPI-1020 (FIG. 2A), 0.44 mg/ml budesonide v. 0.63 mg/ml of TPI-1020 (FIG. 2B), various doses of S-nitroso-N-acetylpenicillamine (SNAP) (FIG. 2C), specific doses of TPI-1020, budesonide, and salbutamol compared to a combination of salbutamol plus TPI-1020 or salbutamol plus budesonide (FIG. 2D), specific doses of TPI-1020, budesonide, and salbutamol compared to a combination of budesonide plus SNAP, TPI-1020 Salbutamol, and budesonide plus salbutamol plus SNAP (FIG. 2D), and 20 mg/ml salbutamol; 0.1 mg/ml TPI-1020; 0.44 mg/ml budesonide (equimolar to 0.1 mg/ml TPI-1010); and 0.7 mM SNAP (equimolar to 0.1 mg/ml TPI-1020); as well as combinations of 20 mg/ml salbutamol plus 0.1 mg/ml TPI-1020; 0.44 mg/ml budesonide plus 0.7 mM SNAP; 20 mg/ml salbutamol plus 0.44 mg/ml budesonide; and 20 mg/ml salbutamol plus 0.44 mg/ml budesonide plus 0.7 mM SNAP (FIG. 2D).

FIG. 3 shows the duration of the protective effect of salbutamol and TPI-1020 against bronchoconstriction induced by histamine, as measured by specific airway conductance (sG_{aw}). Results are shown for treatments given 30, 60 or 90 minutes before histamine challenge.

FIGS. 4A and 4B show the responsiveness of the airways to treatment with formoterol on bronchoconstriction induced by histamine, as measured by specific airway conductance (sG_{aw}). The results show % sG_{aw} for animals treated with either the vehicle or: various doses of formoterol (FIG. 4A), a specific dose of formoterol, various doses of TPI-1020, and combinations of the specific dose of formoterol plus the various doses of TPI-1020 (FIG. 4B).

FIGS. 5A through 5C show the responsiveness of the airways to treatment with tiotropium on bronchoconstriction induced by methacholine, as measured by specific airway conductance (sG_{aw}). The results show % sG_{aw} in animals treated with either the vehicle or: various doses of tiotropium (FIG. 5A), various doses of TPI-1020 (FIG. 5B), and specific doses of tiotropium or TPI-1020 compared to a combination of those specific doses of tiotropium plus TPI-1020 (FIG. 5C).

### DETAILED DESCRIPTION OF THE INVENTION

In one aspect, the invention herein relates to the improvement of the therapeutic performance, i.e., improved pharmacotherapeutic efficacy when compared with the prior art products, of a bronchodilator when administered by inhalation in combination with the NO-donating steroidal compounds as contemplated by the present invention.

The term NO-donating steroidal compound as set forth herein refers to a compound that has a steroidal backbone with a nitric oxide (“NO”)-donating moiety attached thereto. Under certain physiological conditions these compounds are able to donate, release and/or directly or indirectly transfer any of the three redox forms of nitrogen monoxide (NO⁺, NO⁻, NO.), such that: a) the biological activity of the nitrogen monoxide species is expressed at the intended site of action, and/or; 2) endogenous production of nitric oxide in vivo is stimulated, and/or; 3) endogenous levels of nitric oxide are elevated in vivo, all the foregoing being further described in U.S. Pat. Nos. 7,282,519 and 7,244,753 the entireties of which are hereby incorporated herein by reference thereto.

It will be appreciated by those skilled in the art that the terms nitrosylated compounds or moieties and nitrosated compounds or moieties refer to substitution with at least one NO or NO₂ group, respectively. The term “nitro” refers to the group NO₂ thus “nitrosated” refers to compounds that have been substituted therewith. The term “nitroso” refers to the group NO thus “nitrosylated” refers to compounds that have been substituted therewith. “Thionitrate” refers to —S—NO₂. “Thionitrite” and “nitrosothiol” refer to —S—NO. “Nitrile” and “cyano” refer to —CN. Generally, nitric oxide donating groups can be added through, for example but not limited thereby, one or more sites such as oxygen (hydroxyl condensation), sulfur (sulfhydryl condensation) and/or nitrogen.

Known methods for nitrosating and/or nitrosylating compounds are described in U.S. Pat. Nos. 5,380,758, 5,859,053, 5,703,073, 6,297,260, and 6,696,592 the disclosures of each of which are incorporated by reference herein in their entirety, as well as in WO 94/03421, WO 94/04484, WO 94/12463, WO 95/09831, WO 95/19952, WO 95/30641, WO 97/27749, WO 98/09948, WO 98/19672, WO 98/21193, WO 00/51988, WO 00/61604, WO 00/72838, WO 01/00563, WO 01/04082, WO 01/10814, WO 01/12584, WO 01/45703, WO 00/61541, WO 00/61537, WO 02/11707, WO 02/30866 and in Oae et al, Org. Prep. Proc. Int., 15(3):165-198 (1983). The methods of nitrosating and/or nitrosylating the steroid compounds described in these references can be applied by one skilled in the art to produce any of the nitrosated and/or nitrosylated compounds described herein. In addition, methods of nitrosating and/or nitrosylating the bronchodilators are described or readily discerned and can be applied by one skilled in the art to produce any of the nitrosated and/or nitrosylated bronchodilator compounds described herein.

Additionally, the present invention relates to lower than expected systemic levels of an inhaled steroid in patients clinically indicated for treatment of respiratory diseases using steroid therapy. Inhaled steroids are known to cause a number of systemic adverse effects. One adverse effect is the suppression of the function of the HPA (hypothalamic-pituitary-adrenal) axis. This suppression is linked to side effects such as growth suppression in children and reduction in bone density in adults. This undesirable HPA axis suppression is typically measured by a reduction in urinary cortisol excretion.

The effect on the HPA axis is directly linked to the level of steroid in the systemic circulation whereby higher levels typically result in more suppression. Systemic steroid load is typically reflected in patient AUC data wherein higher AUC levels correlate with higher levels of systemic steroid. The steroids as contemplated for use in the present invention provide for reduced systemic steroid levels while advantageously achieving a therapeutic respiratory effect with a reduction in undesirable systemic side effects such as HPA axis suppression which may be indicated by reduced free urinary cortisol levels. This results in a safer steroid with a potent therapeutic effect

These compounds as used in accordance with the present invention (including their corresponding stereoisomers, salts, solvates, esters, hydrates, polymorphs, prodrugs, and analogues thereof) are NO donating steroid derivatives derived from the corticosteriod class having the same general ring structure as cortisol, shown below.

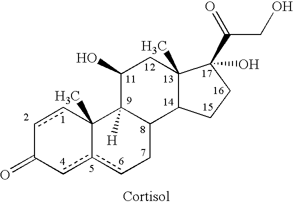

This class of compounds is exemplified by, but not limited to, the following steroids which may be used to form the corticosteroidal backbone of the compounds contemplated for use in accordance with the present invention: 21-acetoxypregnenolone, alclometasone, algestone, amcinonide, beclomethasone, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

It will be further appreciated that while the structures of the foregoing steroid family all approximate the ring structure of cortisol, various structural differences may occur from compound to compound with respect to the existing functional groups attached to the rings, especially at the C-11, C-16, and C-17 positions, as illustrated by some of the well known compounds set forth below. Accordingly, the steroidal backbone to which the NO donating moiety is attached may have multiple sites for attachment based upon the steroid selected as well as the availability of such sites present thereupon.

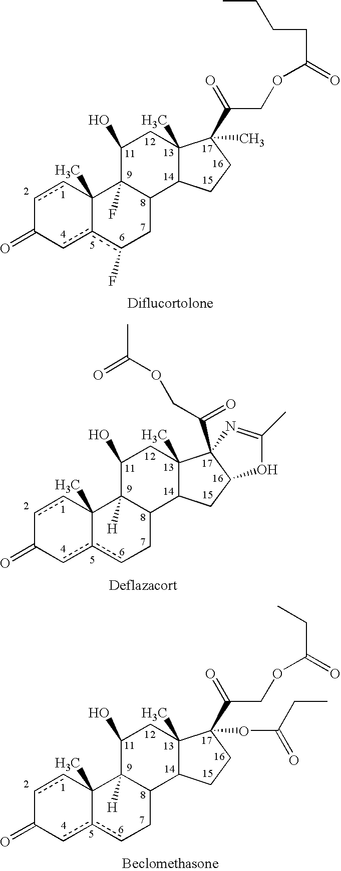

In accordance with the present invention, the steroid backbone is preferably substituted with a NO-donating compound (see for example, 4′-nitrooxymethylbenzoic acid, shown below) through various well known synthesis pathways readily familiar to those skilled in the art.

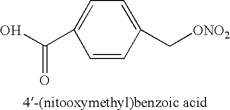

For example, U.S. Pat. No. 6,696,592, which is hereby incorporated herein by reference thereto, illustrates various pathways by which nitrooxyalkylbenzoic acid substituents can be attached to a steroid backbone at various hydroxyl sites present on a steroid, most preferably those hydroxyls positioned either directly or indirectly off the C-11 or the C-17 carbons.

As previously mentioned, the site for attachment of the NO-donating moiety will of course depend upon the structure of the steroidal backbone that is selected as a recipient for such attachment. Further, it will be appreciated by the artisan that the site for attachment must also be chemically conducive to substitution with respect to such considerations as steric hindrances, bond formation constraints, synthesis limitations, and the like. Moreover, there may be simultaneous substitution with the same or a different NO donating moiety off such multiple sites thereby rendering a multiple NO donating effect for the resulting compound, providing however that the character of the sites as well as the moieties to be substituted therewith are chemically conducive to such a multiple substitution, the identification and appreciation of which will be readily apparent to one skilled in the art.

By way of further example, it will be readily apparent from the foregoing benzoic acid structure that the linkage must be facilitated through its carboxylic terminus, thereby rendering the NO donating portion of the structure chemically unobstructed and available to perform a NO donating function while the carboxylic terminus of the NO donating moiety forms an ester linkage with the steroid.

Therefore, in one embodiment, the general structure of the NO-donating compounds as contemplated for use in accordance with the present invention is predicated upon the formation of an ester linkage between the parent steroid and the NO donating moiety. Such a linkage can be directly adjacent to the ring structure as would be the case where the steroidal backbone has an existing hydroxyl group bonding directly to a carbon situated within the ring or, alternatively the linkage can be formed more distal to the ring structure. In either case, the linkage will be cleavable in vivo via hydrolase mediated hydrolysis, in this case esterase mediated hydrolysis, to render a dissociation of the parent steroid and the NO donating moiety that is optimal for maximizing therapeutic effects of both portions of the molecule.

Examples of such cleavable linkers are exemplified by, but in no way limited to, esters, amides, carbamates, and carbonates that are cleavable via in vivo hydrolysis to yield a NO donating moiety and the parent steroid. In one embodiment, as exemplified immediately below, the cleavable site is placed immediately distal to the C-21 hydroxyl group, however, the site of cleavage can be located more distal to the steroid backbone than the C-21 position provided that the portion of the moiety remaining attached thereto subsequent to cleavage does not impede either the steroid's or the NO donating group's therapeutic functionality.

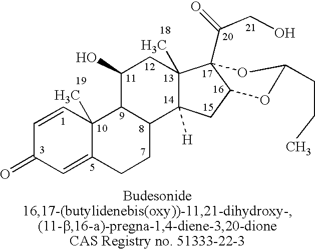

As mentioned in the case of 4′-nitrooxymethylbenzoic acid set forth above, the NO-donating moiety is linked to the steroid by way of its terminal carboxyl group which is available to undergo an esterfication reaction with any of several available functional groups, preferably hydroxyls, which may be present on the parent steroid backbone. It will readily understood that these groups, the identification and availability of which will be immediately apparent to the artisan, must have the appropriate steric and chemical bonding character necessary for stable ester bond formation.

For example, in addition to U.S. Pat. No. 6,696,592 mentioned above, U.S. Pat. Nos. 5,837,698, 5,792,758 and 5,985,862 (all three of which are hereby incorporated herein by reference thereto) describe multiple hydroxyl attachment sites and synthesis methods that can be adapted for constructing compounds made in accordance with the present invention.

In yet another embodiment, the molecule is not cleaved in vivo but rather stays intact thus rendering the cleavable site optional. In such embodiments, the cleavable portion of the linking structure (an ester, amide, or carbamate linkage, for example) is either non-existent or otherwise chemically protected. In the case of the foregoing, it will be appreciated by those skilled in the art that any such linking structure must be compatible with the therapeutic functionality of both the NO donating group as well as the parent steroid compound. Appropriate considerations regarding the same would include but are not limited to issues such as overall size, molecular weight, solubility, steric hindrances, and the like.

As previously mentioned, the present invention is directed to a novel combination therapy wherein a bronchodilator and a nitroderivatized steroid as contemplated herein are administered simultaneously or sequentially to treat respiratory disease in patients, such as relieving bronchoconstriction in patients suffering from chronic pulmonary disease such as asthma and COPD.

Some examples of these nitroderivatized compounds that can be coupled with the bronchodilators of the present invention are exemplified below as having the following general structure, Formula 3:

A-W 3

wherein A is a corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide; and

wherein W is any nitric oxide (“NO”) donating moiety attached thereto capable of donating, releasing and/or directly or indirectly transferring any of the three redox forms of nitrogen monoxide (NO⁺, NO⁻, NO.) that is linked to the steroid backbone by a linking structure that is optionally cleavable in vivo by hydrolase mediated hydrolysis for the treatment of respiratory disease associated with inflammation in a patient in need thereof.

More particularly, W is selected from one of Formulae A-G as further described as follows:

Formula A compounds being represented by: —C(O)-L-X_{O})—(X₁)—NO₂ (as described in published U.S. Patent Application Publication No. 2006/0052594 which is hereby incorporated herein by reference thereto) where L is defined as:

(CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;

X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and

X₁ is a bivalent-linking group selected from the following:

### Y_{AR1}

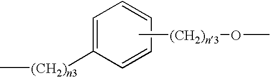

wherein n3 is an integer from 0 to 5 and n3′ is an integer from 1 to 3;

### Y_{AR2}

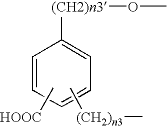

wherein n3 and n3′ have the above meaning or

### Y_{P}

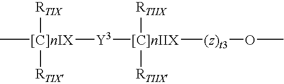

wherein:

nIX is an integer from 0 to 10, preferably 1-3;
nIIX is an integer from 1 to 10, preferably 1-5;
R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
t3 is zero or 1;
Z has the following meaning:

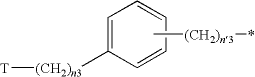

wherein:

shows the position of the ONO₂ group;

T has the following meanings:
—COX₃—, —X₃CO—, wherein X₃═S or X_{O} as above defined;
—X₃— as above defined;
n3 and n′3 are as above defined.

In one preferred embodiment, Y³ is selected from the following bivalent radicals:

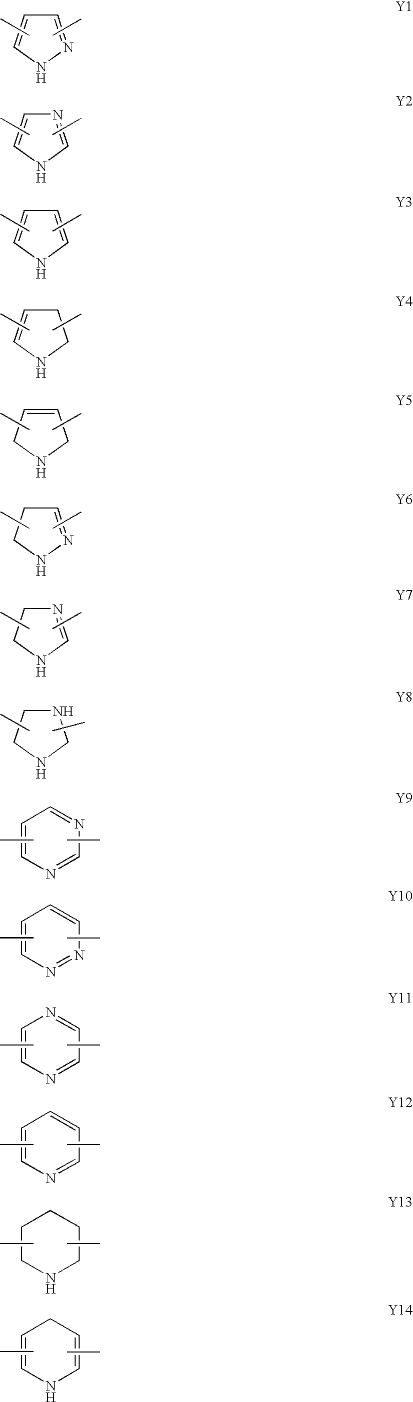

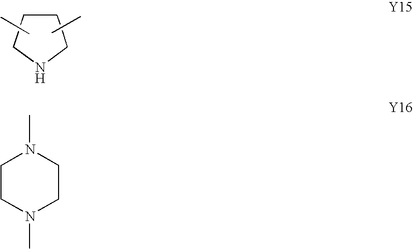

The following are preferred embodiments of Y³: (Y12), having the two free valences in the ortho positions with respect to the nitrogen atom; (Y16) with the two valences linked to the two heteroatoms, (Y1) (pyrazol) 3,5-disubstituted; (Y16) is also preferred.

In one preferred embodiment, L is na=n′b=1, n′a=2, n″b=n′″b=n″a=nb=0, R₄═CH₃, R₅═R_{4″}═R_{5″}═H. The precursors of the bivalent radicals X₁ (as above defined wherein the oxygen free valence is saturated with H and the free valence of the end carbon atom is saturated either with a carboxylic or hydroxyl or amminic group) are commercial products or they can be synthesized in accordance with known methods of the prior art.

The aforementioned compounds having a structure in accordance with Formula A of the present invention can be readily prepared with materials and methods well-known in the art, and particularly as disclosed in published U.S. Patent Application Publication No. 2006/0052594 the entirety of which is hereby incorporated herein by reference thereto.

More particularly, a first variation of the compounds of Formula A can be represented by:

—(CO-L)ₜ-(X)ₜ₁—X₁—NO₂

(as described in U.S. Pat. No. 6,610,676 which is hereby incorporated herein by reference) where t and t1 are integers=1 and L and X are defined as above as L and X₀, but where

X₁ is a bivalent-connecting bridge is selected from the group consisting of Y—O and Y₁, wherein:

for Y—O, Y is a linear or whenever possible branched C₁-C₂₀ alkylene, preferably having from 2 to 5 carbon atoms, or an optionally substituted cycloalkylene having from 5 to 7 carbon atoms; and

for Y₁, Y is selected from Y_{AR1, YAR2}, and Y_{P}, as defined above, and is more particularly:

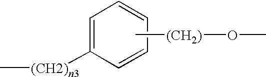

where n3 is an integer from 0 to 3;

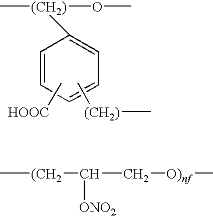

where nf′ is an integer from 1 to 6, preferably from 2 to 4; or

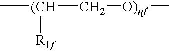

where R_{1f}═H, CH₃ and nf is an integer from 1 to 6, preferably from 2 to 4.

The aforementioned compounds of this first variation can be readily prepared and used with materials and methods well-known in the art, and particularly as disclosed in issued U.S. Pat. No. 6,610,676, the entirety of which is hereby incorporated herein by reference.

A second variation of the compounds of Formula A can be represented as:

—(CO-L)ₜ-(X)ₜ₁—X₁—NO₂

(as described in U.S. Pat. Nos. 7,056,905, 7,205,288, 7,196,075, 7,160,871, and 7,157,450 all of which being hereby incorporated herein by reference) where t and t1 are integers=1 and L is defined as above;

wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;

X is equal to O, C═O, NH, NR_{1c} where R_{1c} is a C₁-C₁₀, and preferably a C₁-C₄ linear or branched alkyl; OH, CH₃, Cl, N(—CH₂—CH₃)₂, SCH₂F, SH,

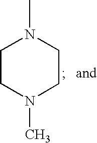

X₁ is a bivalent-connecting bridge is selected from the group consisting of Y—O and Y₁ as defined above.

The aforementioned compounds having a structure in accordance with this second variation of Formula A of the present invention can be readily prepared and used with materials and methods well-known in the art, and particularly as disclosed in issued U.S. Pat. Nos. 7,056,905, 7,205,288, 7,196,075, 7,160,871, and 7,157,450, and U.S. Patent Application Publication No. 2007/0238882, which are hereby incorporated herein by reference in their entirety.

A most preferred embodiment of a compound of Formula A is represented as

C(O)-L-(X₀)—(X₁)—NO₂

wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, n″a, n′b, n″b and n′″b are 0 (thus L is CH₂O.)

### X₀ is C═O, and

X₁ is the bivalent-linking group Y_{AR1}, preferably

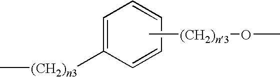

Formula B compounds being represented by: —C(O)CH₂O—X_{Z} (as described in U.S. Pat. No. 7,297,808 which is hereby incorporated herein by reference thereto) or alternatively by —C(O)CH₂OC(O)—X_{Z} wherein X_{Z} is defined as follows:

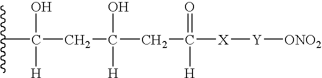

and X is O, S, NH or NHR₁, where R₁ is a straight or branched alkyl with 1 to 10 carbon atoms, preferably CH₃; and
Y is a bivalent radical having the following meanings a)-h)
a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl)-ONO₂ or is —O(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃
b)

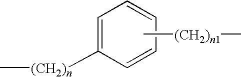

c)

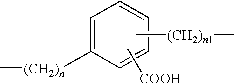

wherein for b) and c) above n is an integer from 0 to 20, and n¹ is an integer from 0 to 20;
d)

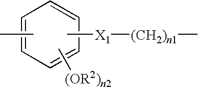

wherein, n¹ is as defined above and n is an integer from 0 to 2; X₁ is —OCO— or —OCO— and R₂ is H or CH₃;
e)

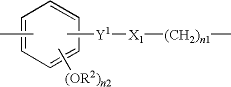

wherein n¹, n², R₂ and X₁ are as defined above; and Y¹ is either —CH₂—CH₂— or —CH₂═CH₂—(CH₂)ₙ₂—;
f)

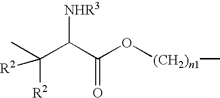

wherein n¹ and R² are as defined above; R³ is H or COCH₃; with the proviso that when Y is selected from the bivalent radicals mentioned under b) through f), the, —ONO₂ group is bound to —(CH₂)ₙ₁;
g)

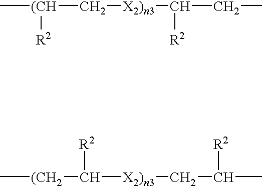

wherein X₂ is O or S, n³ is an integer from 1 to 6, preferably from 1 to 4, and R² is defined above;
h)

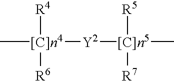

wherein: n⁴ is an integer from 0 to 10; n⁵ is an integer from 1 to 10; R⁴, R⁵, R⁶, and R⁷ are the same or different, and are H or straight or branched C₁-C₁₀ alkyl; and preferably R⁴, R⁵, R⁶, and R⁷ are H; wherein the —ONO₂ group is bound to the following structure:

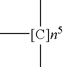

wherein, n⁵ is defined above;
Y² is a heterocyclic saturated, unsaturated or aromatic 5 or 6 members ring, containing one or more heteroatoms selected from nitrogen, oxygen, sulfur, and is selected from the following structures H1 through H13:

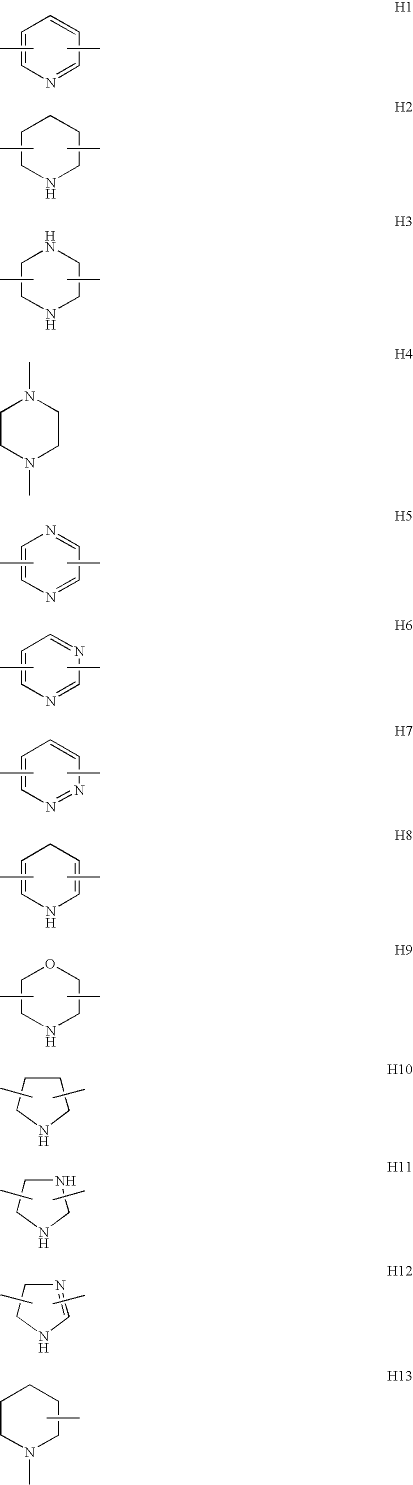

The aforementioned compounds having a structure in accordance with Formula B of the present invention can be readily prepared and used with materials and methods well-known in the art, and particularly as disclosed in issued U.S. Pat. No. 7,297,808 the entirety of which is hereby incorporated herein by reference thereto.

Formula C compounds being represented by: —C(O)CH₂O—(X₁—ONO₂)_{S} (as described in U.S. Pat. No. 7,217,733 which is hereby incorporated herein by reference thereto) or alternatively by —C(O)CH₂OC(O)—(X₁—ONO₂)_{S} wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—. The aforementioned compounds having a structure in accordance with Formula C of the present invention can be readily prepared and used with materials and methods well-known in the art, and particularly as disclosed in the aforementioned patent the entirety of which is, as previously stated, hereby incorporated herein by reference thereto.

Formula D compounds being represented by: —C(O)CH₂O—B—C) (as described in U.S. Pat. No. 7,199,258 which is hereby incorporated herein by reference thereto) wherein X₁ is defined as follows:

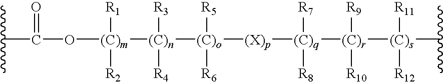

where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above.

The aforementioned compounds having a structure in accordance with Formula D of the present invention can be readily prepared and used with materials and methods well-known in the art, and particularly as disclosed in issued U.S. Pat. No. 7,199,258 the entirety of which is hereby incorporated herein by reference thereto.

Formula E compounds being represented by: —C(O)CH₂O—B—C^{Z}, various compounds of which are disclosed and/or exemplified in U.S. Pat. No. 5,837,698 as well as U.S. Pat. Nos. 5,792,758 and 5,985,862 (all three of which are hereby incorporated herein by reference thereto) C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO donating portion of the compound, C^{Z}, via an amide, ester, carbamate or carbonate linkage that is induced adjacent to the 21 position.

In accordance with the various embodiments of the present invention, one embodiment provides using compounds of Formula H wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans.

Alternatively, R₁ can be selected from any of the following chemical moieties that have been appropriately substituted with a NO-donating group: lower alkyls/alkenyls/alkynyls; that are substituted or unsubstituted (excepting out the necessary substitution with the NO donating group as required in accordance with the present invention, the proviso being applicable in all the remaining groups recited below in this paragraph); substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.

In yet another embodiment, B—C^{Z} is equal to the following structure:

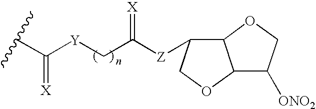

wherein, n is an integer from 1 to 4; X═O or S; Y=methylene, O, or NH₂; and Z=O or NH₂.

In another embodiment, the cleavable site is located more distal to the steroid backbone than in Formula E, provided that the portion of the moiety that remains attached to the steroid subsequent to cleavage does not impede either the steroid's or the NO donating group's therapeutic functionality. In accordance with such an embodiment, W=Formula E′ where E′ is —C(O)CH₂O—(B′—C^{Z}) wherein B′ is a spacer preferably containing 12 carbon atoms or less that connects the steroid backbone at the hydroxy immediately adjacent to the C-21 position with the NO donating portion of the compound, C^{Z}, via an amide, ester, carbamate or carbonate linkage that is not adjacent to the C-21 position.

In accordance with the various embodiments of the present invention, one embodiment provides using compounds of Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans.

Alternatively, R₁ can be selected from any of the following chemical moieties that have been appropriately substituted with a NO donating group: lower alkyls/alkenyls/alkynyls; that are substituted or unsubstituted (excepting out the necessary substitution with the NO donating group as required in accordance with the present invention, the proviso being applicable in all the remaining groups recited below in this paragraph); substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.

In accordance with the various embodiments of the present invention, another further embodiment provides using compounds of Formula E or E′ with the alternative structures readily discerned from U.S. Pat. Nos. 5,837,698, 5,792,758 and 5,985,862 the entireties of which are hereby incorporated herein by reference thereto. The aforementioned compounds having structures in accordance with Formula E or E′ of the present invention can be readily prepared with materials and methods well-known in the art, and particularly as disclosed in the aforementioned patents.

Formula F compounds being represented by: —C(O)CH₂O—K (as described in U.S. Pat. No. 7,282,519 which is hereby incorporated herein by reference thereto) or alternatively by —C(O)CH₂OC(O)—K wherein K is defined as follows:

—Y—(CR₄R_{4′})ₚ-T-(CR₄R_{4′})ₚ—ONO₂;

—Y—(CR₄R_{4′})ₒ-[phenyl]-T-(CR₄R_{4′})ₚ—ONO₂;

wherein T is ortho, meta or para;

—Y—B-[piperazinyl]-W—(CR₄R_{4′})ₚ—ONO₂;

—Y—(CR₄R_{4′})ₚ—V—B-T-(CR₄R_{4′})ₚ—ONO₂;

—Y—(CR₄R_{4′})ₚ-T-C(O)—(CR₄R_{4′})ₚ—(CH₂)—ONO₂;

—Y—(CR₄R_{4′})ₚ—C(Z)-(CH₂)_{q}-T-(CR₄R_{4′})_{q}—(CH₂)—ONO₂;

—Y—(CR₄R_{4′})ₚ-T-(CH₂)_{q}—V—(CR₄R_{4′})_{q}—(CH₂)—ONO₂;

—Y—(CR₄R_{4′})ₚ—V—(CH₂)_{q}—V—(CR₄R_{4′})_{q}—(CH₂)—ONO₂;

—Y—(CR₄R_{4′})ₒ—(W)_{q}—(CH₂)_{q}—V—(CR₄R_{4′})ₒ—(CH₂)—ONO₂;

—NRⱼ—O—(CH₂)ₒ—V—(CR₄R_{4′})_{q}—(CH₂)—ONO₂;

—NRⱼ—O—(CH₂)ₒ—(W)_{q}—(CR₄R_{4′})_{q}—(CH₂)—ONO₂;

—O—NRⱼ—(CH₂)ₒ—(W)_{q}—(CR₄R_{4′})_{q}—(CH₂)—ONO₂;

In accordance with the various embodiments of the present invention, another further embodiment provides using compounds of Formula F with the alternative structures readily discerned from U.S. Pat. No. 7,282,519 which is, as previously stated, hereby incorporated herein by reference thereto.

The aforementioned compounds having structures in accordance with Formula F of the present invention can be readily prepared with materials and methods well-known in the art, and particularly as disclosed in the aforementioned patent.

Formula G compounds being represented by: —C(O)CH₂O—X1 (as described in U.S. Pat. Nos. 7,256,205 and 7,244,753 which are both hereby incorporated herein by reference thereto) or alternatively by —C(O)CH₂OC(O)—X1 wherein X1 is defined as follows:

—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;

or alternatively, X1 is equal to K which is further defined as:

—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);

where, —(U)—(V) is equal to —Wᵢ-Eⱼ-W_{g}—[C—(Rₑ)(R_{f})]_{z}-T-Q;

or alternatively U and V are taken independently wherein U is O, S, or —N(Rₐ)(Rᵢ) and V is nitro, nitroso, or hydrogen;

wherein, a, b, c, d, g, i and j are each independently an integer from 0 to 3;

p, x, y and z are each independently an integer from 0 to 10;
W is independently —C(O)—, —C(S)—, -T-, —[C—(Rₑ)(R_{f})]ₕ—, an alkyl group, an aryl group, a heterocyclic ring, an arylheterocyclic ring, or —(CH₂CH₂O)_{q}—;
E at each occurrence is independently -T-, an alkyl group, an aryl group, —[C—(Rₑ)(R_{f})]ₕ—, a heterocyclic ring, an arylheterocyclic ring, or —CH₂CH₂O)_{q}—;
h is an integer form 1 to 10;
q is an integer of from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;
k is an integer from 1 to 3;
T at each occurrence is independently a covalent bond, a carbonyl, an oxygen, —S(O)ₒ— or —N(Rₐ)(Rᵢ);
O is an integer from 0 to 2;
Rₐ is a lone pair of electrons, a hydrogen or an alkyl group;
Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ-T-Q, then the “-T-Q” subgroup designated as X can be a hydrogen, an alkyl, an alkoxy, an alkoxyalkyl, an aminoalkyl, a hydroxy, a heterocyclic ring or an aryl group.

In cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and [C—(Rₑ)(R_{f})]₂ denotes —C(Rₑ)(R_{f})—C(Rₑ)(R_{f})—.

In accordance with the various embodiments of the present invention, another further embodiment provides using compounds of Formula G with the alternative structures readily discerned from U.S. Pat. Nos. 7,256,205 and 7,244,753 which are, as previously stated, hereby incorporated herein by reference thereto.

The aforementioned compounds having structures in accordance with Formula G of the present invention can be readily prepared with materials and methods well-known in the art, and particularly as disclosed in the aforementioned patents.

It will be appreciated by those skilled in the art that further alternative embodiments of the foregoing compounds as contemplated for use in the present invention may be readily prepared using the various NO-donating groups set forth in U.S. Pat. Nos. 7,238,814, 7,235,237, 7,186,753, 7,186,708, 7,160,920, 7,122,539, 7,087,588, 6,987,120, 6,909,007, 6,869,974, 6,828,342, 6,794,372, 6,469,065, 6,645,965, 6,635,273, 6,613,784, 6,593,347, 6,579,863, 6,465,463, 6,433,182 6,417,207, 6,197,762, 6,143,734, 6,043,232, 5,780,495, 5,621,000, 5,189,034, RE37,116 as well as U.S. Patent Application Pub. Nos. 20070248676, 20070238740, 20070197499, 20070112194, 20070072854, 20070060586, 20070010571, 20060154905, 2006000943, 20050176694, 20040082652, and 200462243.

Although W is shown above as being linked at C-17 of the steroidal moiety A, it would be understood, and is expressly disclosed herein, that W can also be linked at C-11 of steroidal moiety A, wherein W is as defined above, including the alternative Formulae A-G. Moreover, the NO-donating group may be linked at C-21 of a corticosteroid having a C-21 present.

In accordance with the present invention, the aforementioned compounds are delivered to the patient in conjunction with a bronchodilator. Treatment involves the delivery of the needed drugs to the pulmonary system. The drugs delivered to the lung are of two types: a bronchodilator, such as an anticholinergic or beta-agonist, and the NO-donating compounds set forth above in order to potentiate the pharmacotherapeutic efficacy of the brochodilator while retaining its anti-inflammatory properties.

More particularly, in one embodiment the invention provides a pharmaceutical composition comprising a therapeutically effective amount of (i) at least one bronchodilator, or a physiologically acceptable salt or solvate thereof, and (ii) at least one of the aforementioned NO-donating compounds for treating respiratory disease associated with inflammation in a patient in need thereof.

In another embodiment, the invention provides a method for treating respiratory disease associated with inflammation in a patient in need thereof, comprising the simultaneous or sequential administration of a therapeutically effective amount of (i) at least one bronchodilator, or a physiologically acceptable salt or solvate thereof, and (ii) at least one of the aforementioned NO-donating compounds.

In yet another embodiment, the invention provides for methods of using a therapeutically effective amount of (i) at least one bronchodilator, or a physiologically acceptable salt or solvate thereof, and (ii) at least one of the aforementioned NO-donating compounds for treating respiratory disease associated with inflammation in a patient in need thereof.

In still another embodiment, the invention provides a method for the use of a therapeutically effective amount of (i) at least one bronchodilator, or a physiologically acceptable salt or solvate thereof, and (ii) at least one of the aforementioned NO-donating compounds in the manufacture of a medicament for treating respiratory disease associated with inflammation in a patient in need thereof.

In one embodiment, the respiratory disease is a chronic pulmonary disease. In another embodiment, the chronic pulmonary disease is selected from pulmonary obstructive conditions such as asthma or chronic obstructive pulmonary disease such as emphysema or chronic bronchitis. In another embodiment, the respiratory diseases may also include eosinophilic cough, bronchitis, sarcoidosis, pulmonary fibrosis, pulmonary hypertension, cystic fibrosis, and bronchiectasis.

The term “sequential administration” in the context of the present invention refers to the administration of the at least one bronchodilator and at least one of the aforementioned NO-donating compounds in separate doses, wherein these doses are administered to the patient less than one hour apart from one another. More preferably, the bronchodilator and the NO-donating compound are administered to the patient in separate doses within 30 minutes of one another.

Typical anticholinergic bronchodilators include tiotropium and ipratropium. Representative β₂-agonists for use in the present invention may include salbutamol (known as albuterol in the United States), bitolterol mesylate, formoterol, isoproterenol, levalbuterol, metaproterenol, salmeterol, terbutaline, and fenoterol. There are three distinct groups of beta-adrenoreceptors: β₁, β₂ and β₃, which are classically identified in cardiac, airway smooth muscle, and adipose tissue, respectively. In the context of the present invention which relates to the treatment of respiratory disease, the terms “beta-adrenoreceptor” and “beta-receptor” refer to the β₂-adrenoreceptor, and the term “beta-agonist” refers to β₂-agonists. The molecular mechanisms of β₂-adrenoreceptors have been well documented, and are reviewed in Johnson, M. in J Allergy Clin Immunol. 2006 January; 117(1):18-24, the contents of which are herein incorporated by reference.

Like all G protein-coupled receptors, the β₂-receptor has 7 transmembrane-spanning α-helices. There are 3 extracellular loops, with one being the amino-terminus, and 3 intracellular loops, with a carboxy-terminus. The receptor is N-glycosylated; these modifications are important for insertion into the cell membrane and for agonist-induced receptor trafficking. One cysteine of the human β₂-receptor is palmitoylated, acting to anchor the carboxy-terminus to the membrane. Autoradiographic studies of human lung have suggested that β₂-adrenoreceptors are widely distributed, occurring not only in airway smooth muscle (30-40,000 per cell) but also on other cells in the lung, such as epithelial and endothelial cells, type II cells, and mast cells. However, the receptor density on the latter cells is substantially lower than in smooth muscle.

There are two forms of the β₂-adrenoreceptor, activated and inactivated. Under resting conditions, these 2 forms are in equilibrium, with the inactivated state being predominant. The β₂-adrenoreceptor is in the activated form when it is associated with the α-subunit of the Gs protein, together with a molecule of guanosine triphosphate (GTP). The replacement of the GTP by guanosine diphosphate dramatically reduces the affinity of the α-subunit for the receptor, causing dissociation and inducing the receptor to return to its low energy inactivated form.

It is accepted that β₂-adrenoreceptor activation is mediated by increased intracellular cAMP levels. This is the result of stimulation of adenylate cyclase, which catalyzes the conversion of adenosine triphosphate into cAMP. The coupling of the β₂-adrenoreceptor to adenylate cyclase is affected through a trimeric Gs protein, consisting of an α-subunit (which stimulates adenylate cyclase) and βγ-subunits (which transduce other signals). cAMP levels are then regulated through the activity of phosphodiesterase isozymes/isoforms, which degrade it to 5′-AMP.

The molecular structure of a β₂-agonist determines the manner in which it interacts with the β₂-adrenoreceptor. Short-acting β₂-agonists, such as albuterol, which are hydrophilic in nature, access the active site of the β₂-adrenoreceptor directly from the extracellular aqueous compartment. There is therefore a rapid onset of action. However, these drugs rapidly re-equilibrate, their residency time at the receptor active site is limited, and the resulting duration of action is short (4-6 hours).

Formoterol is an example of a long acting β₂-agonist. Formoterol is moderately lipophilic, and it is taken up into the cell membrane in the form of a depot, from where it progressively leaches out to interact with the active site of the β₂-adrenoreceptor. The size of the depot is determined by the concentration or dose of formoterol applied. The onset of action of formoterol is somewhat delayed compared with that of albuterol, and the duration of activity is longer and concentration dependent. This profile has been confirmed clinically in asthmatic patients in whom bronchodilation was observed for 8, 10, and 12 hours after doses of 6, 12, and 24 μg, respectively.

Salmeterol, on the other hand, is more than 10,000 times more lipophilic than albuterol. The molecule partitions rapidly (<1 minute) into the cell membrane and then diffuses laterally to approach the activity site of the β₂-adrenoreceptor through the membrane, with this process appearing to be slow (>30 minutes). The onset of action of salmeterol in airway smooth muscle is therefore slower than that of other β₂-agonists, such as albuterol and formoterol. The mechanism of action of salmeterol involves the interaction of the side chain of the molecule with an auxiliary binding site (exosite), a domain of highly hydrophobic amino acids within the fourth domain of the β₂-adrenoreceptor. Salmeterol appears to be inherently long acting, in that its effects are independent of dose as a result of exosite binding.

The affinity of a ligand is a measure of the avidity of its binding to its receptor. Few 2-agonists have been shown to have much higher affinity than isoproterenol, and indeed, albuterol has a relatively low affinity for β₂-adrenoreceptor. In contrast, salmeterol and formoterol have 3- to 4-fold higher affinities for the β₂-adrenoceptor than albuterol.

Different β₂-agonists share a common role in that they stimulate β₂-adrenoreceptors in the autonomic system to open the airways by relaxing the muscles around the airways that may tighten during bronchospasms, and relieve dyspnea.

In addition to β₂-agonists and anticholinergics, other bronchodilating compounds may be used in the present invention. These may include other drugs such as ephedrine and xanthines. Representative xanthines include Theophylline, Aminophylline, and Oxtriphylline. Xanthines are typically not delivered by inhalation, but can be administered in other ways (orally, intramuscularly, intravenously, or by suppositories).

In some instances, the desired dosage form is intended for use by patients with severe conditions. The invention is also directed to a regimen of dosing that maintains the appropriate levels of the drugs and is administered in a form that a patient with a weakened condition can achieve the intended dosage during a single delivery session. The required drugs can be relatively long-acting such that delivery of the drug does not require an unreasonable regimen of the patient with respect to the portion of the day which must be dedicated to the delivery of the therapy.

In the present invention, a vehicle by which they may be mixed and co-administered is required. The vehicle could comprise a variety of pharmaceutically acceptable vehicles for delivery of the drugs by inhalation of a solution or suspension or of a dry powder or suspension. As mentioned below, formulations for administration by inhalation can be prepared for use as aerosolized medicaments such as in manner recited herein or, alternatively, in U.S. Pat. No. 5,458,135 and U.S. Pat. No. 5,447,150, which are hereby incorporated herein by reference thereto.

In another embodiment of the present invention the bronchodilator, such as salbutamol, and at least one of the aforementioned NO-donating compounds may be provided in a variety of pharmaceutically acceptable vehicles, including, but not limited to a solution of 30% DMSO, 30% ethanol and 40% saline.

In another embodiment of the present invention, the inhalation drug comprises a therapeutically effective amount of a bronchodilator and the NO-donating compound. By “therapeutically effective” amount is meant a nontoxic but sufficient amount of a compound to provide the desired therapeutic effect, which in the present case, would be that dose of bronchodilator and the aforementioned NO-donating compound effective to relieve, ameliorate, or prevent symptoms of the condition or disease being treated, e.g. respiratory disease such as asthma and COPD.

In another embodiment, the at least one bronchodilator is one bronchodilator selected from a β₂-agonist and an anticholinergic.

In another embodiment, the bronchodilator is a β₂-agonist. In yet another embodiment, the β₂-agonist is selected from the group consisting of salbutamol, bitolterol mesylate, formoterol, isoproterenol, levalbuterol, metaproterenol, salmeterol, terbutaline, and fenoterol. In still another embodiment, the β₂-agonist is a short-acting β₂-agonist, such as salbutamol. In yet another embodiment, the β₂-agonist is a long-acting β₂-agonist, such as formoterol.

In another embodiment, the bronchodilator is an anticholinergic, such as tiotropium and ipratropium.

In still another embodiment, the at least one bronchodilator is two bronchodilators comprising a β₂-agonist and an anticholinergic. In another embodiment, the β₂-agonist component is selected from the group consisting of salbutamol, bitolterol mesylate, formoterol, isoproterenol, levalbuterol, metaproterenol, salmeterol, terbutaline, and fenoterol, and the anticholinergic component is selected from the group consisting of tiotropium or ipratropium.

In one embodiment, the NO-donating compound is administered in a daily dose from about 100 μg to 4800 μg. In another embodiment, the daily dose is from about 100 μg to 2400 μg.

Acceptable doses of bronchodilators for use in the present invention are known to those of skill in the art. For instance, the anticholinergic tiotropium, which is sold under the trade name Spiriva™, may be administered in powder form from a soft mist inhaler at a dose of about 5 to 30 μg per day.

The anticholinergic ipratropium, which is sold under the trade name Atrovent™, may be administered via a metered-dose inhaler at a dose of about 10 μg to 320 μg four times per day, preferably about 40 μg to 80 μg four times per day (up to about 1280 μg total daily dose). Ipratropium solutions for nebulization are generally administered to adults at a dose of about 60 μg to 2 mg four times per day, preferably about 250 μg to 500 μg four times per day (up to about 8 mg total daily dose), and are administered to children at a dose of about 30 μg to 1 mg four times per day, preferably about 125 μg to 250 μg four times per day (up to about 4 mg total daily dose).

Acceptable doses of β₂-agonists are also known to those of skill in the art.

Formoterol, as sold under the trade names Oxeze™ or Foradil™, may be administered via dry powder inhaler or metered-dose inhaler at a dose of about 1 μg to 100 μg twice daily, preferably about 6 μg to 24 μg twice daily (up to about 200 μg total daily dose).

Salmeterol, as sold under the trade name Serevent™, may be administered via dry powder inhaler or metered-dose inhaler in a dose of about 10 μg to 200 μg twice daily, preferably about 50 μg twice daily (up to about 400 μg total daily dose).

Salbutamol, as sold under the trade name Ventolin™, may be administered via dry powder inhaler or metered-dose inhaler in doses of about 25 μg to 800 μg four times daily, preferably about 100 μg to 200 μg four times daily (up to about 3.2 mg total daily dose). If administered via nebulizer, the acceptable dose is from about 300 μg to 20 mg four times daily, preferably about 1.25 mg to 5 mg four times daily (up to about 80 mg total daily dose).

Acceptable doses of other bronchodilators such as ephedrine and xanthines are also known to those of skill in the art. For instance, ephedrine may be administered at a dose of about 6 mg to 100 mg, preferably about 25 mg, four times per day (up to about 400 mg per day). As noted above, xanthines may be administered orally (liquid form, long-acting or short-acting pill form), intramuscularly, intravenously, or by suppositories. Theophylline may be administered in a dose of about 25 mg to 5 g per day, preferably about 100 to 1200 mg per day. Aminophylline may be administered in a dose of about 25 mg to 6 g per day, preferably about 100 mg to 1500 mg per day. Oxtriphylline may be administered in a dose of about 25 mg to 6.5 g per day, preferably 100 to 1600 mg per day.

Suitable weight ratios of bronchodilator to the aforementioned NO-donating compounds for use in inhaled formulations may be determined using the foregoing measurements. For instance, the weight ratio of NO-donating compound to salbutamol in the compositions for administration by inhalation via dry powder inhaler or metered-dose inhaler according to the present invention is preferably within the range of about 1:8 to 4:1.

Suitable physiologically acceptable variations (including the corresponding pharmaceutically acceptable stereoisomers, salts, solvates, esters, hydrates, polymorphs, prodrugs, and analogues thereof) of the foregoing bronchodilators and NO-donating compounds may be used in the present invention and are known to those of skill in the art.

For example, suitable physiologically acceptable salts may include acid addition salts derived from inorganic and organic acids, such as the hydrochloride, hydrobromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, salicylate, acetate, succinate, lactate, glutarate, gluconate, tricarballylate, hydroxynaphthalenecarboxylate or oleate.

The inhalation drug of the present invention may also be provided in sterile, unit dose treatments. Moreover, the sterile formulation of the present invention is expected to provide a stable inhalation solution such that the formulation can be stored (e.g. on a shelf) for long periods of time.

In one embodiment, at least one bronchodilator and at least one of the aforementioned NO-donating compounds are administered simultaneously.

In another embodiment, at least one bronchodilator and TPI-1020 at least one of the aforementioned NO-donating compounds are administered sequentially.

In one embodiment, the intended dose regimen is one to four times daily administration, or administration as needed by a patient, by inhalation from a dry powder inhaler or metered-dose inhaler, where the suitable daily dose of salbutamol is in the range of about 100 μg to about 3.2 mg, preferably about 400 μg to 800 μg, and the suitable daily dose for TPI-1020 is about 100 μg to 2400 μg, with a preferred daily dose of about 100 μg to 1600 μg.

A person of skill in the art will understand that the particular dose used will strongly depend on the method of administration (type of inhaler, oral dosage form vs. inhalation, etc.), the patient (age, weight etc) and the severity of the disease (mild, moderate, severe asthma etc).

For administration by inhalation, the compositions according to the invention may be conveniently delivered by conventional means such as in the form of a pressurized metered dose inhaler prepared in a conventional manner or in combination with a spacer device such as the Volumatic™ (Glaxo Group trade mark) device. In the case of a metered dose inhaler, a metering valve is provided to deliver a metered amount of the composition. Spray compositions may for example be formulated as aqueous solutions or suspensions and may be inhaled from a nebuliser. Aerosol spray formulations may also be used, for example in which the active ingredients are suspended, optionally together with one or more stabilisers, in a propellant. The most frequently used chlorofluorocarbon propellants are trichloromonofluoromethane (propellant 11), dichlorodifluoromethane (propellant 12), dichlorotetrafluoroethane (propellant 114), tetrafluoroethane (propellant 134a) and 1,1-difluoroethane (propellant 152a). Low concentrations of a surfactant such as sorbitan trioleate, lecithin, disodium dioctylsulphosuccinate or oleic acid may also be used to improve the physical stability. The two drugs may be administered separately in similar ways.

Alternatively, for administration by inhalation or insufflation, the compositions according to the invention may take the form of a dry powder composition, for example a powder mix of the active ingredients and a suitable carrier such as lactose. The powder compositions may be presented in unit dosage form in, for example, capsules, cartridges or blister packs from which the powder may be administered with the aid of an inhaler such as the Rotahaler™ inhaler or Turbuhaler™ inhaler, or in the case of blister packs by means of the Diskhaler™ inhaler.

A diluent or carrier, generally non-toxic and chemically inert to the medicament e.g. lactose, dextran, mannitol or glucose or any additives that will give the medicament a desired taste, can be added to the powdered medicament.

Non-adherence medication therapy and medication error are considerable problems. These problems can be significantly reduced by providing patients a prepackaged, premixed, premeasured amount of bronchodilator and the NO-donating compound. Providing these compounds in this fashion makes therapy simple because it increases convenience and eliminates confusion in preparing appropriate dosages.

In another embodiment, the present invention also comprises a device for use in the relief of symptoms associated with respiratory diseases such as asthma and COPD, including bronchospasm.

In an alternative embodiment, the system and/or kit of the present invention comprises an inhalation formulation comprising a therapeutically effective amount of at least one bronchodilator and at least one NO-donating compound in a prepackaged, premeasured, premixed and/or single unit dose form for the treatment of asthma or COPD.

The present invention also provides a process for making a prepackaged, premeasured, premixed, sterile inhalation formulation comprising a single unit dose of a therapeutically effective amount of at least one bronchodilator and at least one NO-donating compound.

In another embodiment, the bronchodilator and the NO-donating compound may be administered via routes other than inhalation. For example, salbutamol may be orally administered to adults in a liquid formulation in amounts from about 0.5 mg to 16 mg, preferably about 2 mg to 4 mg, three to four times per day (up to about 64 mg total daily dose). In the case of children ages 2 to 6 years, the acceptable dosage of salbutamol by this mode of administration may be estimated at 0.1 mg/kg/day. Salbutamol is also available in slow release pill form, and may be administered in a dose of about 0.5 mg to 16 mg twice daily, preferably about 2 mg to 4 mg twice daily (total daily dose of up to about 32 mg). Terbutaline may be orally administered in pill form in a dosage of about 1 mg to 20 mg, preferably about 5 mg, three to four times daily (total daily dose of up to about 80 mg), or in a slow release oral dosage form in an amount of about 2 mg to 30 mg, preferably about 7.5 mg, twice daily (total daily dose of up to about 60 mg).

Accordingly, the present invention provides methods for the use of these compounds in a corresponding pharmaceutical formulation together with one or more pharmaceutically acceptable carriers and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), and topical (including dermal, buccal, and sublingual) administration although the most suitable route may depend upon for example the condition and disorder of the recipient. The formulations may conveniently be presented in unit dosage form and may be prepared by any of the methods well known in the art of pharmacy.

All methods include the step of bringing into association a preparation or a compound as defined above, including the corresponding pharmaceutically acceptable stereoisomers, salts, solvates, esters, hydrates, polymorphs, prodrugs, and analogues thereof (“active ingredient”), with the carrier which constitutes one or more accessory ingredients. In general, the formulations are prepared by uniformly and intimately bringing into association the active ingredient with liquid carriers or finely divided solid carriers, or both, and then, if necessary, shaping the product into the desired formulation.

Formulations as contemplated for use with the present invention that are suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Tablets may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain antioxidants, buffers, bacteriostats and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

Formulations for administration by inhalation can be prepared for use as an aerosolized medicament, such as in manner recited above as well as in U.S. Pat. No. 5,458,135 and U.S. Pat. No. 5,447,150, which are hereby incorporated herein by reference thereto.

Preferred unit dosage formulations are those containing an effective dose, as hereinbelow recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The NO-donating compounds as contemplated for use in accordance with the present invention may be administered generally from about 100 ug to as much as 1 gram per day depending upon the method of administration as well as other clinical factors well known to those skilled in the art. For example, dosages for oral administration can range from about 100 ug to about 250 mg per day while dosages administered via intravenous methods may range from about 100 ug to about 1 g per day however the preferable route of administration is via inhalation where the dosages range from about 100 ug to about 2500 ug per day.

The compounds as used with in conjunction with the present invention are preferably administered by inhalation, and as a second alternative, orally or by injection (intravenous or subcutaneous). The precise amount of compound administered to a patient will be the responsibility of the attendant physician. However, the dose employed can depend on a number of factors, including the age and sex of the patient, the precise disorder being treated, and its severity. Also, the route of administration may vary depending on the condition and its severity.

Advantageously, it has been discovered that the use of a NO-modified steroid or a NO-donating compound in the presence of a steroid (preferably a corticosteroid) can potentiate or synergistically affect the action of a bronchodilator, e.g., a beta-agonist or a muscarinic antagonist, or the like. This advantageous use can allow for the reduction of the dose of one or more of the active pharmaceutical ingredients (APIs) used in the combination, or can enhance the efficacy of one or both APIs as compared to the efficacy of that same dose when not used in the combinations as described herein.

### EXAMPLES

The following examples describe experimental work conducted to demonstrate the advantageous potentiation or synergism exhibited by the combined administration and combination compositions in accordance with the subject invention.

All the experiments described in these Examples set forth below were conducted using the following materials and methods or experimental protocols.

### Materials and Methods

### Animals

Groups of six male Dunkin-Hartley guinea-pigs, weighing 350-450 g were used throughout. Animals received food and water ad libitum and lighting was maintained in the room (22+/−2° C.) on a 12 h cycle. This work complied with the guidelines for the care and use of laboratory animals according to the Animals (Scientific Procedures) Act 1986.

### Measurement of Respiratory Function

Whole body plethysmography of the conscious guinea-pig was used to monitor airway function, recorded as specific airways conductance (sG_{aw}). The method was as described by Griffiths-Johnson et al. (J. Pharmacol. Meth. 1988; 11:233-240), although a computerized data acquisition system replaced the original oscilloscope and angle resolver (Danahay et al. Clin. Exp. Allergy 1997; 28:513-522). Animals were fitted with a face mask and placed in a restrainer which was then slid into the plethysmograph chamber. The computer ran AcqKnowledge software using a Biopack data acquisition system. This system was capable of acquiring and storing data referring to the air across a pneumotachograph (Mercury FIL) as the animal breathed. The resulting change in box volume (pressure) was simultaneously measured.

Changes in air flow and box pressure were measured using UP1 and UP2 pressure transducers, respectively. The resulting waveforms could then be rapidly analysed by comparing the gradients of the flow and the box pressure waves at a point where flow tended towards zero, i.e. end tidal flow. A function of these two parameters, allowing for air pressure and the weight of the animal, gave the resulting value for specific airway conductance (sG_{aw}). A minimum of five breaths were analysed for each animal at each time point to arrive at a mean value, which is the reported value.

In the experimental examples described herein, the reported values for sG_{aw} measurements were taken following histamine or anticholenergic challenge without treatment. This pre-treatment sG_{aw} value measured after histamine or methacholine challenge, by convention, determined 100% bronchoconstriction for the animal. Following treatment with the experimental agents, including agents which are part of the subject invention, sG_{aw} values (level of bronchoconstriction) were reported as a percentage of the pre-treatment value. Thus, a 10% post-treatment sG_{aw} value, as compared to the 100% pre-treatment sG_{aw}, is a 90% reduction in bronchoconstriction, or 90% reduction of airway hyperresponse (AHR). A 20% post-treatment sG_{aw} value corresponds to an 80% reduction in bronchoconstriction or AHR. Thus, the lower the post-treatment sG_{aw} value, the better the reduction of bronchoconstriction and AHR. For purposes of the subject invention, the more desired response is a lower sG_{aw} value (less histamine- or methacholine-induced bronchoconstriction, and less AHR).

Before each experiment, the animals were handled and familiarized with the apparatus to reduce stress. Between recordings, animals were removed from the plethysmograph and placed in a holding cage.

### Spasmogen Exposures

Baseline sG_{aw} values were taken on Day 1 and Day 2 30 minutes prior to exposure to 3 mM histamine for 20 seconds. Changes in % sG_{aw} were measured 0, 5, 10 minutes after each histamine exposures.

### Drug/Vehicle Exposures

On day 2, animals were treated by inhalation exposure to TPI-1020 (0.1, 0.3, and 0.63 mg/ml), salbutamol (3, 10, 20 and 30 ug/ml; salbutamol hemisulfate salt from Sigma-Aldrich (Product No. S5013)), budesonide (0.44 mg/ml; equimolar dose to 0.63 mg/ml TPI-1020), or vehicle (DMSO 30%, ethanol 30%, saline 40%) for 15 minutes. All drugs or vehicle were delivered as a whole body exposure chamber. Fifteen minutes later, animals were exposed to bronchoconstriction agents, e.g., histamine or methacholine, and measurements of sG_{aw} were taken.

### Example 1

### TPI-1020 Potentiates the Bronchodilating Effect of Salbutamol

This example relates to the synergistic or potentiating effect of the combination of TPI-1020 and salbutamol for the relief of bronchospasm induced by histamine in guinea pigs.

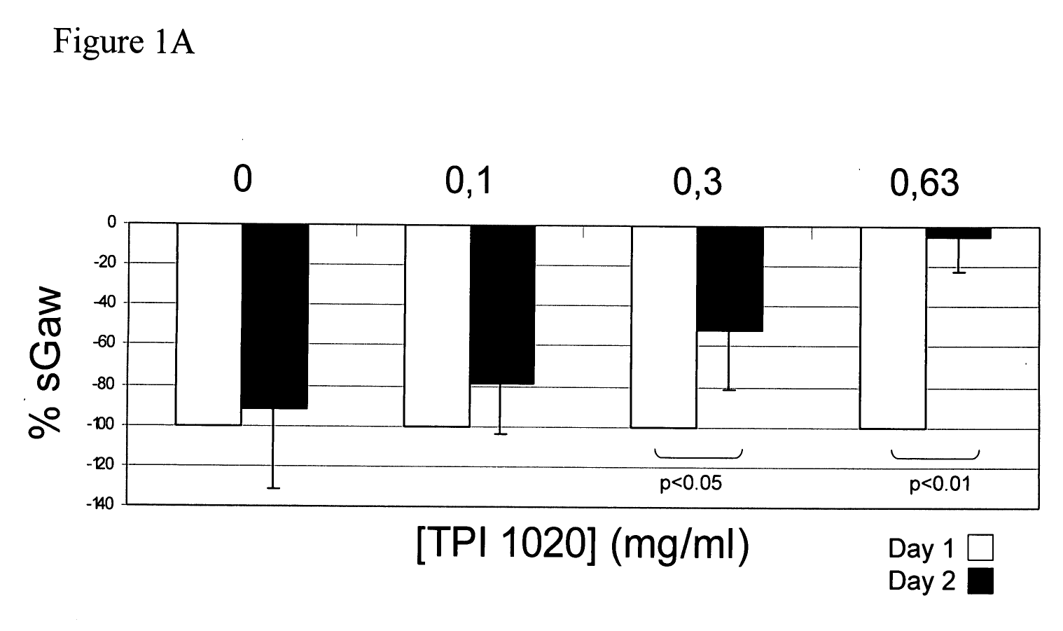

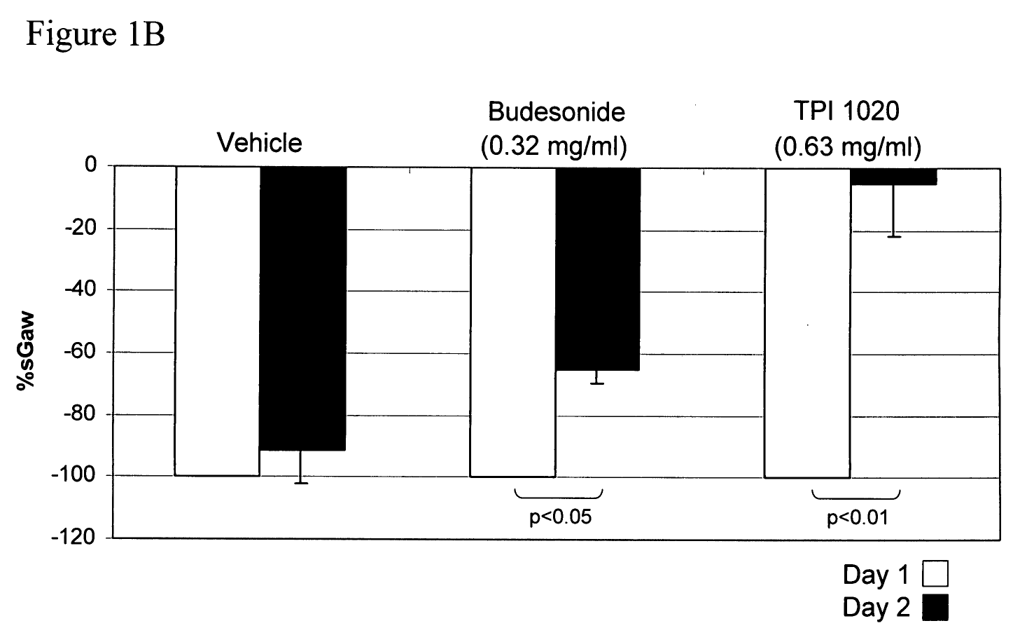

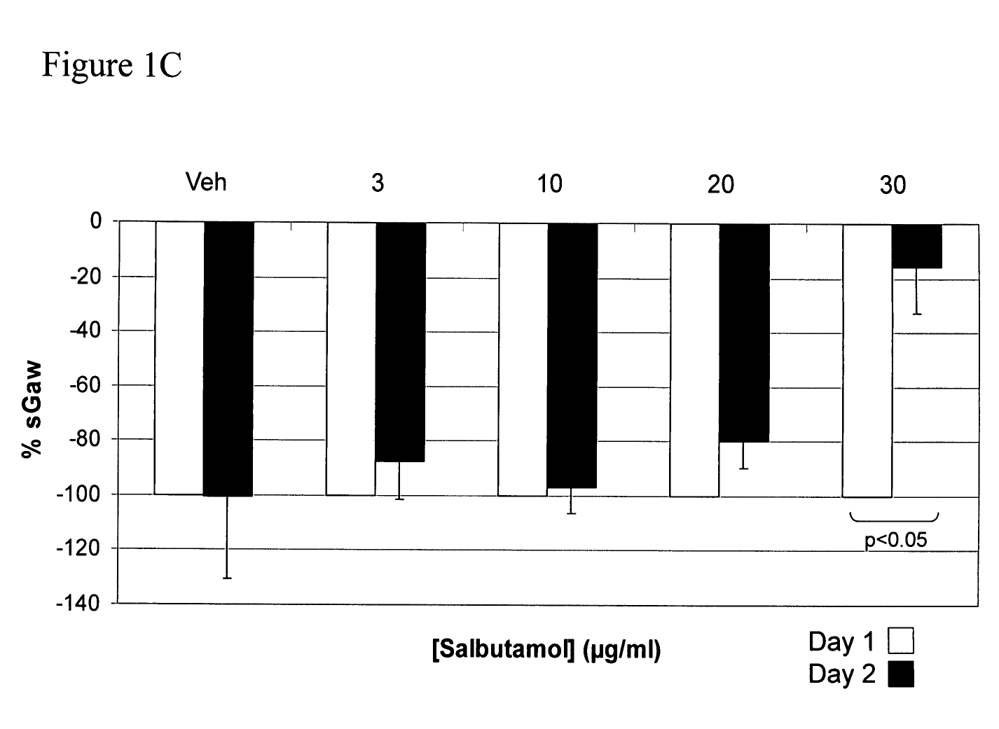

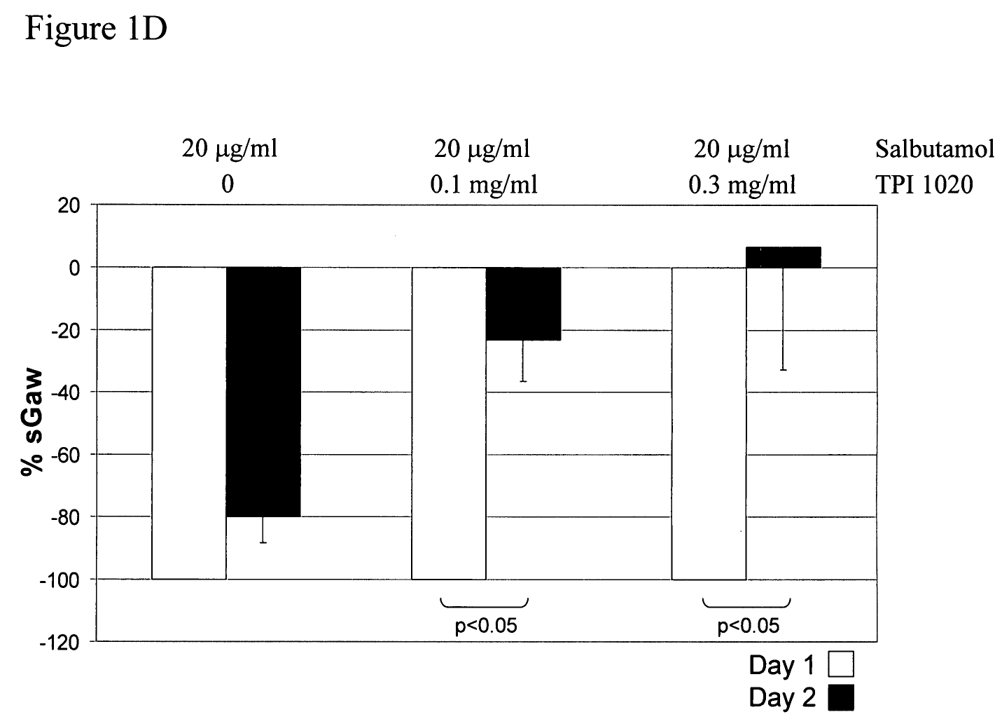

Results in FIG. 1A depict the effect of various doses of TPI-1020 at relieving histamine-induced bronchospasm in guinea pigs, as measured by specific airway conductance (sG_{aw}). Baseline readings were taken on Day 1 and on Day 2, 30 minutes prior to histamine challenge (3 mM). Following histamine exposure, sG_{aw} values were measured to determine 100% bronchoconstriction or AHR. On Day 2, animals were treated with either the vehicle (“0” amount of active) or a particular dose of TPI-1020 (0.1 mg/ml, 0.3 mg/ml, or 0.63 mg/ml), 15 minutes prior to histamine challenge. The sG_{aw} for each dose was determined from recordings made at 0, 5 and 10 minutes after the histamine exposure, and the average sG_{aw} were determined. The sG_{aw} values for Day 2 are expressed as a percentage inhibition of bronchoconstriction compared to Day 1 sG_{aw} values (100%). Each point or reported value represents the mean+/−S.E.M. (n=6). Statistical significance was determined for p<0.05 using Student's paired t-test.

As shown in FIG. 1A, treatment with 0.1 mg/ml of TPI-1020 resulted in 80% sG_{aw}, or a 20% inhibition of the airway hyperresponse (AHR) (no significant inhibition), 0.3 mg/ml of TPI-1020 resulted in 52% sG_{aw} (48% inhibition of the AHR to histamine) (p<0.05), while AHR was reduced by 95% (5% sG_{aw}) when animals were treated with 0.63 mg/ml of TPI-1020 (p<0.01).

In contrast, as shown in FIG. 1B, an equimolar dose of budesonide (0.44 mg/ml) resulted in only 35% inhibition (p<0.05) of the bronchoconstriction induced by histamine, as compared to the 95% inhibition effect of TPI-1020 at the 0.63 mg/ml dose.

Treatment with salbutamol also resulted with a protective effect to histamine-induced bronchoconstriction. As shown in FIG. 1C, treatment with salbutamol at a dose of 30 μg/ml reduced AHR to histamine by 85% (p<0.05). However, salbutamol had no significant inhibition effect at lower doses of 3, 10 and 20 μg/ml.

When animals were treated with 0.3 mg/ml of TPI-1020 and 20 μg/ml of salbutamol, in combination, AHR was completely abolished (p<0.01) (FIG. 1D). These same doses of each active, alone, showed only 48% and 80% inhibition, respectively (see FIGS. 1A and 1C). Reducing the dose of TPI-1020 to 0.1 mg/ml, in combination to 20 μg/ml of salbutamol, maintained a significant bronchodilating effect (75% inhibition, p<0.05).

These results demonstrated that certain doses of TPI-1020, combined with certain doses of salbutamol, produced a potentiating or synergistic bronchodilating effect by significantly inhibiting bronchoconstriction induced by histamine challenge.

The results of the experiments presented in FIGS. 1A through 1D are presented in Table 1, below.

TABLE 1

| | Pre- | Post- | | |
|---|---|---|---|---|
| | treatment | treatment | | % |
| Treatment/Dose | (% sGaw) | (% sGaw) | SEM | Inhibition |
| FIG. 1A | | | | |
| TPI-1020 (mg/ml) | | | | |
| 0 | −100 | −91.6 | 39.6 | 8 |
| 0.1 | −100 | −78.7 | 24.8 | 21 |
| 0.3 | −100 | −51.9 | 29.5 | 48 |
| 0.63 | −100 | −5.1 | 17.0 | 95 |
| FIG. 1B | | | | |
| Budesonide (equimola | −100 | −65.2 | 4.4 | 35 |
| dose to 0.63 mg/ml of | | | | |
| TPI 1020) | | | | |
| FIG. 1C | | | | |
| Salbutamol (□g/ml) | | | | |
| 0 | −100 | −100.5 | 30.2 | −1 |
| 3 | −100 | −87.6 | 14.0 | 12 |
| 10 | −100 | −96.7 | 9.4 | 3 |
| 20 | −100 | −80.1 | 9.7 | 20 |
| 30 | −100 | −15.6 | 17.2 | 84 |
| FIG. 1D | | | | |
| Salbutamol (20 μg/ml) + | −100 | −23.3 | 13.2 | 77 |
| TPI1020 (0.1 mg/ml) | | | | |
| Salbutamol (20 μg/ml) + | −100 | 6.9 | 39.5 | 107 |
| TPI1020 (0.3 mg/ml) | | | | |

### Example 2

### Bronchodilating Effect of NO-Donor+Steroid+Bronchodilator

This example relates to experiments carried out to demonstrate the synergistic or potentiating effect of the combination of NO-donor, a steroid, and a bronchodilator for the relief of bronchospasm induced by histamine in guinea pigs.

In these experiments, as in Example 1, baseline readings were taken on Day 1 prior to treatment (Pre-treatment) to determine 100% values for histamine-induced bronchoconstriction or AHR. On Day 2, using the vehicle as a control, animals were treated with the vehicle, or the vehicle plus an active or a combination of actives. Average sG_{aw} was determined from recordings of sG_{aw} made at 0, 5 and 10 minutes after the histamine exposure (Post-treatment). The sG_{aw} values for Day 2 were expressed as a percentage of Day 1 sG_{aw} sG_{aw} values, set at 100%. Each point represents the mean+/−S.E.M. (n=6). Statistical significance was determined for p<0.05 using Student's paired t-test.

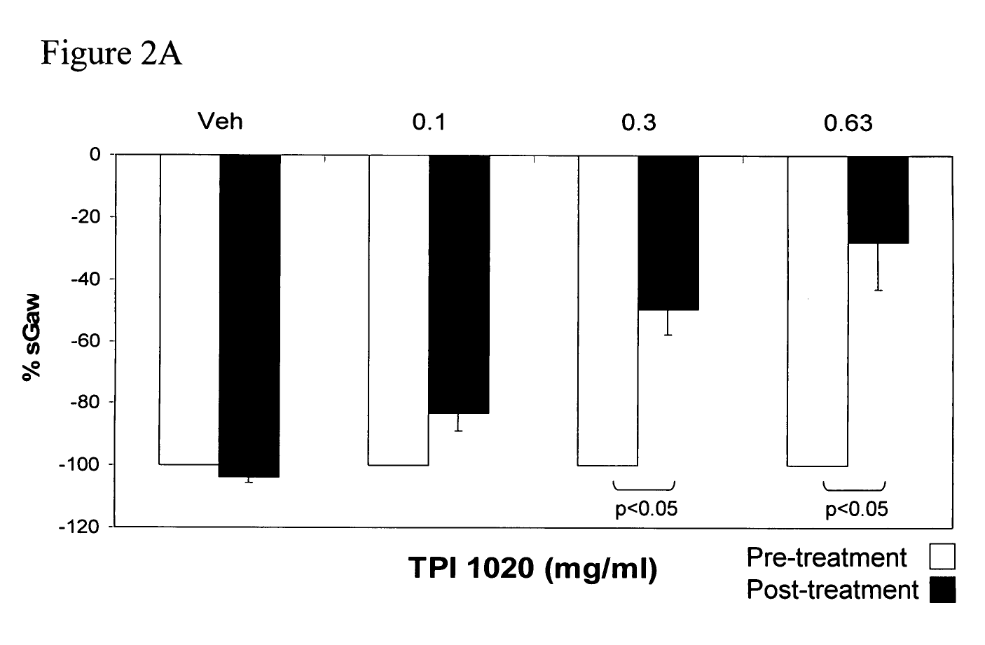

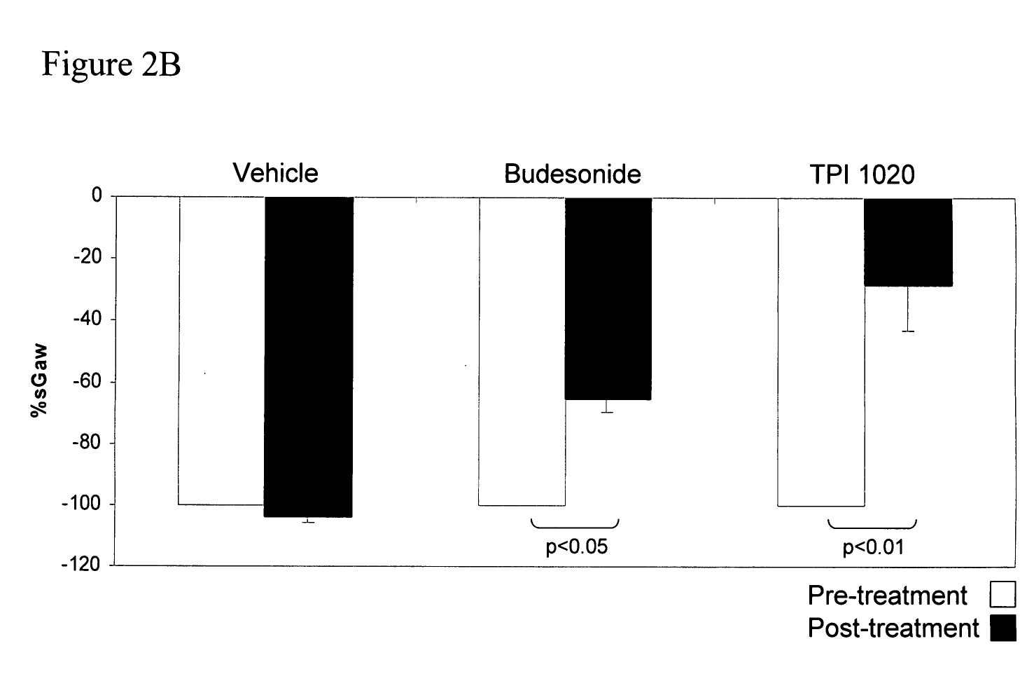

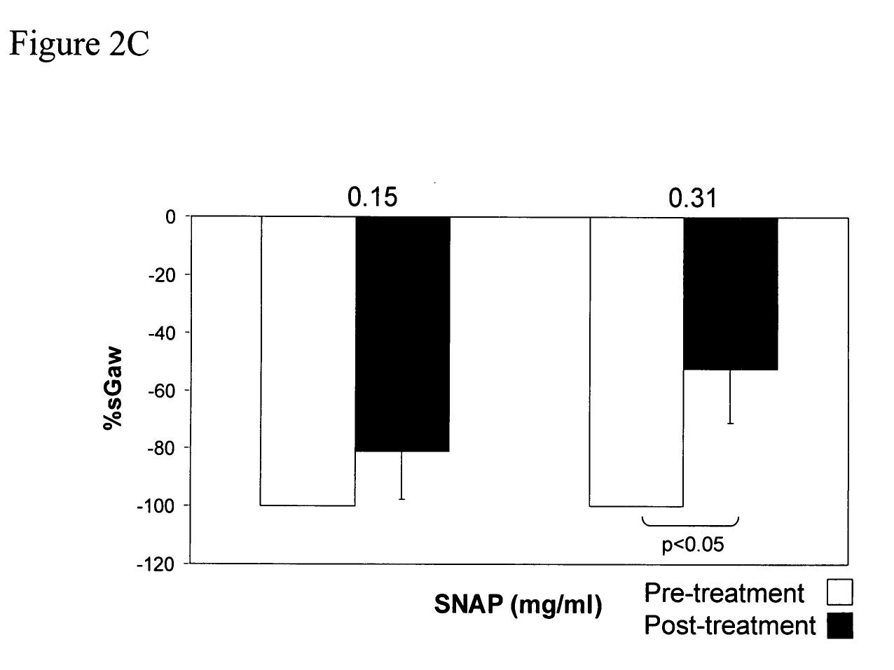

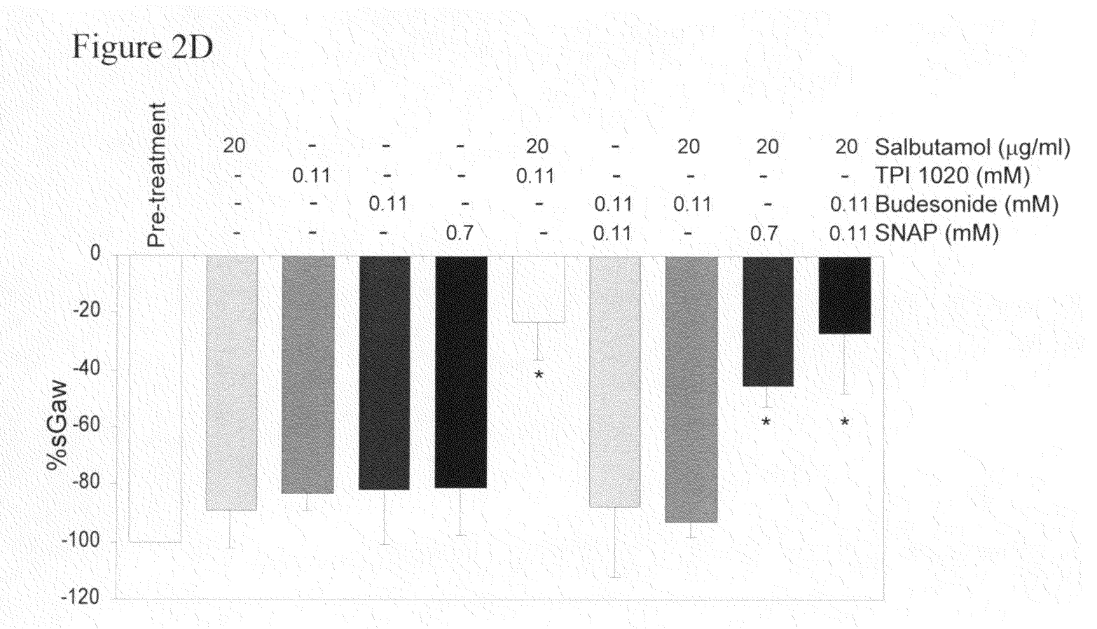

FIGS. 2A through 2D depict the results showing potentiated responsiveness of the airways by treating guinea pigs with a combination of a NO-donor, such as TPI-1020 or SNAP, and a bronchodilator, such as salbutamol, following histamine-induced bronchoconstriction as compared to: NO-donors, alone (e.g., TPI-1020 or S-nitroso-N-acetylpenicillamine (SNAP)); a steroid, alone (e.g., budesonide); a steroid in combination with a bronchodilator (e.g., budesonide+salbutamol); a steroid in combination with a NO-donor (e.g., budesonide+SNAP or budesonide+TPI-1020), or a combination of NO-donor with a bronchodilator in the presence of a steroid (e.g., SNAP+salbutamol+budesonide).

Results in FIG. 2A depict the effect of various doses of TPI-1020 at relieving histamine-induced bronchospasm in guinea pigs, as measured by specific airway conductance (sG_{aw}). Baseline readings were taken on Day 1 and on Day 2, 30 minutes prior to histamine challenge (3 mM) to determine 100% bronchoconstriction or AHR induced by histamine. On Day 2, animals were treated with either the vehicle (“veh”) or a particular dose of TPI-1020 (0.1 mg/ml, 0.3 mg/ml, or 0.63 mg/ml). As shown in FIG. 2A, treatment with 0.1 mg/ml of TPI-1020 resulted in 80% inhibition of the airway hyperresponse (AHR) (no significant inhibition), 0.3 mg/ml of TPI-1020 resulted in 48% inhibition (p<0.01) of the AHR to histamine, while AHR was reduced by 80% when animals were treated with 0.63 mg/ml of TPI-1020 (p<0.01). This inhibition of bronchoconstriction exhibited by TPI-1020, as compared to only 40% reduction of AHR using equimolar amount (0.44 mg/ml) of budesonide, alone (FIG. 2B). The inhibition of AHR (% sG_{aw}) 0.15 and 0.31 mg/ml S-nitroso-N-acetylpenicillamine (SNAP) is shown in FIG. 2C, resulting in 20% and 35% reduction, respectively. Only the use of 0.31 mg/ml showed a statistically significant reduction.

In FIG. 2D, TPI-102 (0.1 mg/ml), 0.7 mg/ml of budesonide, and 20 ug/ml of salbutamol were shown to produce insignificant reduction of AHR. The same amount of budesonide, combined with salbutamol also failed to provide a significant reduction of AHR. These results are compared to 0.1 mg/ml of TPI-1020 plus 20 ug/ml of salbutamol, again demonstrating potentiation or synergy using the combination of salbutamol and TPI-1020. These results also demonstrate that the potentiation or synergy is not a result of the steroid moiety of TPI-1020.

FIG. 2D shows the results of an experiment designed to show that a bronchodilator, such as salbutamol, is potentiated by a NO-donating compound when administered in the presence of a steroid, such as TPI-1020 (a NO-modified corticosteroid) or SNAP (a NO-donor)+budesonide (a corticosteroid). Specifically, FIG. 2D shows that administration of a NO-donor (e.g., TPI-1020), a steroid (e.g., budesonide), or a bronchodilator (e.g., salbutamol), do not significantly reduce AHR when used alone. In addition, a combination of steroid (budesonide) and a NO-donor also do not significantly reduce AHR. However, when a NO-donor and a steroid and a bronchodilator (salbutamol) are combined, a significant decrease in AHR is observed (TPI+Salb) or budesonide in combination with salbutamol and 0.7 mM SNAP. This suggests the three components (bronchodilator/NO-donor/steroid) are effective in significantly reducing AHR in response to histamine.

In further experiments, as shown in FIG. 2D, various combinations of salbutamol, TPI-1020, budesonide and SNAP were compared to pretreatment values (clear bar graph), or each of these agents, alone (bars 2-5 from the left side of the graph). A combination the two agents, salbutamol plus TPI-102, provided the highest reduction of bronchoconstriction (about 80%) upon histamine challenge, whereas a combination of three agents, salbutamol plus budesonide plus SNAP, provided comparable reduction of bronchoconstriction (more than 75% reduction of AHR). Combinations of budesonide plus SNAP and salbutamol plus budesonide provided the least reduction of bronchoconstriction (less than 20% reduction, each). The combination of salbutamol plus SNAP provided a moderate reduction of bronchoconstriction (about 50%).

These results are consistent with the discovery that a preferred treatment, which can unexpectedly provide a synergistic or potentiated reduction in bronchoconstriction upon histamine challenge, results from administration of one or more agents that provide a steroid component, a bronchodilator component, and a NO-donor component. Specifically, these results demonstrate that administration of salbutamol (a bronchodilator), budesonide (a steroid) and SNAP (a NO-donor) provide the potentiated reduction in histamine-induced bronchoconstriction versus any combination of these agents that do not provide each of the steroid, bronchodilator, and a NO-donor components. These results further demonstrate that providing these three components can be achieved when one or more are combined in a single agent. For example, the results confirm that potentiation or synergism are achieved by the combination of salbutamol (a bronchodilator) plus TPI-1020, which provides both a steroid component and a NO-donor component.

The results of the experiments presented in FIGS. 2A through 2D are presented in Table 2, below.

TABLE 2

| | Pre- | Post- | | |
|---|---|---|---|---|
| | treatment | treatment | | % |
| Treatment/Dose | (% sGaw) | (% sGaw) | SEM | Inhibition |
| FIG. 2A | | | | |
| TPI-1020 (mg/ml) | | | | |
| 0 | −100 | −103.8 | 2.0 | −4 |
| 0.1 | −100 | −83.2 | 5.6 | 17 |
| 0.3 | −100 | −49.5 | 8.1 | 50 |
| 0.63 | −100 | −27.9 | 15.3 | 72 |
| FIG. 2B | | | | |
| Budesonide (equimolar dose | −100 | −65.2 | 4.4 | 35 |
| to 0.63 mg/ml of TPI 1020) | | | | |
| FIG. 2C | | | | |
| SNAP (mg/ml) | | | | |
| 0.15 | −100 | −81.2 | 16.4 | 19 |
| 0.31 | −100 | −52.6 | 18.5 | 47 |
| FIG. 2D | | | | |
| Salbutamol (20 μg/ml) | −100 | −89.0 | 13.0 | 11 |
| TPI-1020 (0.11 mMl) | −100 | −83.2 | 5.6 | 17 |
| Budesonide (0.11 mM) | −100 | −81.8 | 19.0 | 18 |
| SNAP (0.7 mM) | −100 | −81.2 | 16.4 | 19 |
| Salbutamol (20 □g/μml) + | −100 | −23.2 | 13.2 | 77 |
| TPI-1020 (0.11 mMl) | | | | |
| Budesonide (0.11 mM) + | −100 | −87.8 | 24.1 | 12 |
| SNAP (0.11 mM) | | | | |
| Budesonide (0.11 mM) + | −100 | −93.0 | 5.2 | 7 |
| Salbutamol (20 μg/ml) | | | | |
| Salbutamol (20 μg/ml) + | −100 | −45.6 | 7.1 | 54 |
| SNAP (0.7 mM) | | | | |
| Salbutamol (20 μg/ml) + | −100 | −27.0 | 21.4 | 73 |
| Budesonide (0.11 mM) + | | | | |
| SNAP (0.11 mM) | | | | |

### Example 3

### Duration of Bronchodilating Effect of TPI-1020+Salbutamol

This example relates to experiments carried out to demonstrate the duration of the synergistic or potentiating effect of the combination of a nitroderivatized corticosteroid (TPI-1020) and a bronchodilator (salbutamol) for the relief of bronchospasm induced by histamine in guinea pigs.

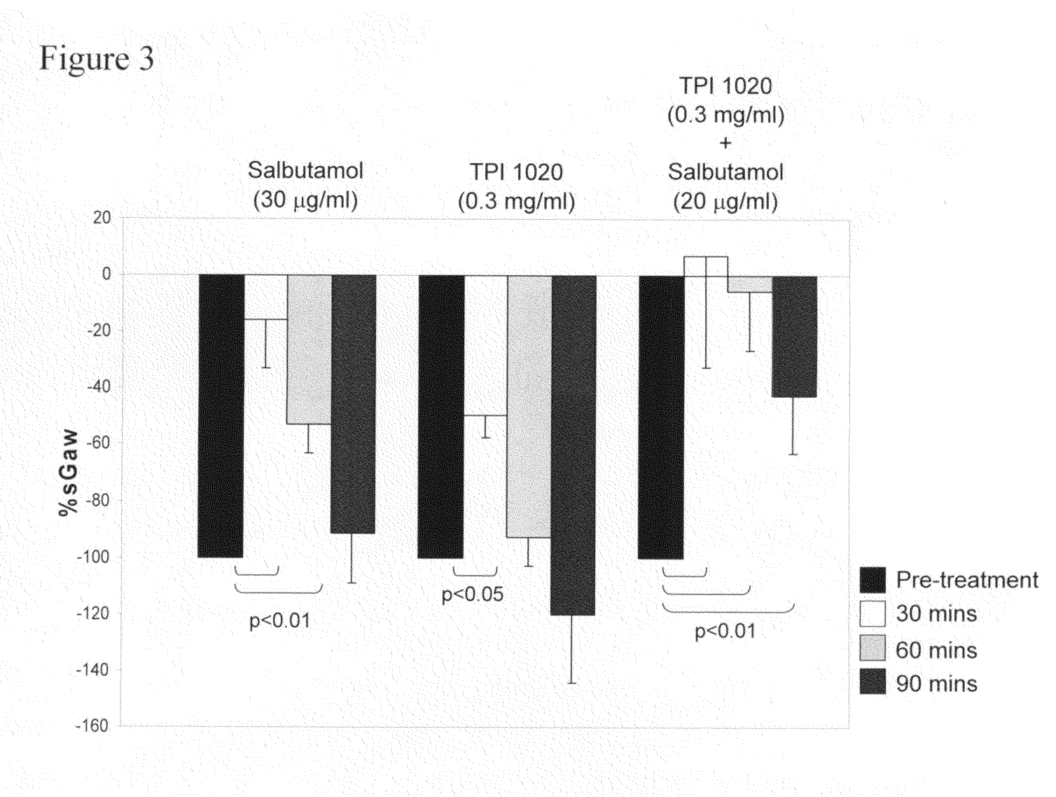

FIG. 3 shows the duration of the protective effect of the combination of a NO-donor (salbutamol) and a NO-donor (TPI-1020) against bronchoconstriction induced by histamine. Baseline readings were taken on Day 1 prior to treatment (Pre-treatment) to determine 100% bronchoconstriction or AHR induced by histamine. On Day 2, animals were treated 30, 60 or 90 minutes before histamine challenge, either with the vehicle, salbutamol, TPI-1020 or with a combination of TPI-1020 and salbutamol. Recordings of sG_{aw} were made at 0, 5 and 10 minutes after the histamine exposure (Post-treatment). The sG_{aw} values of Day 2 are expressed as a percentage of Day 1 sG_{aw} values set at 100%. Each point represents the mean+/−S.E.M. (n=6). Statistical significance was determined for p<0.05 using Student's paired t-test. FIG. 3 shows that the combination treatment using a NO-modified steroid in combination with bronchodilator significantly reduces AHR for at least 90 minutes after treatment, whereas the NO-modified steroid (TPI-1020), alone, significantly reduced AHR only 30 minutes, and a bronchodilator (salbutamol) significantly reduced AHR for only 60 minutes.

The results of the experiments presented in FIG. 3 are presented in Table 3, below.

TABLE 3

| | Pre- | Post- | | |
|---|---|---|---|---|
| Treatment/Dose | treatment (% | treatment (% | | % |
| FIG. 3 | sGaw) | sGaw) | SEM | Inhibition |
| Salbutamol (30 μg/ml) | | | | |
| 30 mins | −100 | −15.6 | 17.2 | 84 |
| 60 mins | −100 | −52.7 | 10.1 | 47 |
| 90 mins | −100 | −91.4 | 17.4 | 9 |
| TPI-1020 (0.3 mg/ml) | | | | |
| 30 mins | −100 | −49.5 | 8.1 | 50 |
| 60 mins | −100 | −92.7 | 10.1 | 7 |
| 90 mins | −100 | −120.1 | 24.2 | −20 |
| TPI-1020 (0.3 mg/ml) + | | | | |
| Salbutamol (20 μg/ml) | | | | |
| 30 mins | −100 | 6.9 | 39.5 | 107 |
| 60 mins | −100 | −5.6 | 20.7 | 94 |
| 90 mins | −100 | −42.7 | 20.5 | 57 |

### Example 4

### Bronchodilating Effect of TPI-1020+Formoterol

This example relates to experiments carried out to demonstrate that TPI-1020 potentiates the action of other bronchodilators in the beta-agonist class, including long-acting beta-agonists (LABAs), by showing the effects of the combination of TPI-1020 and formoterol for the relief of bronchospasm induced by histamine in guinea pigs.

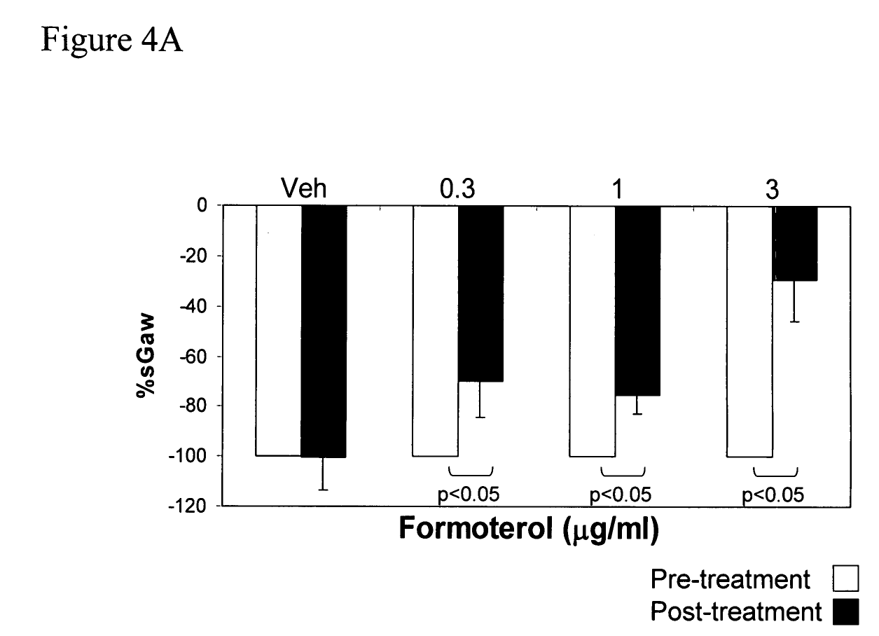

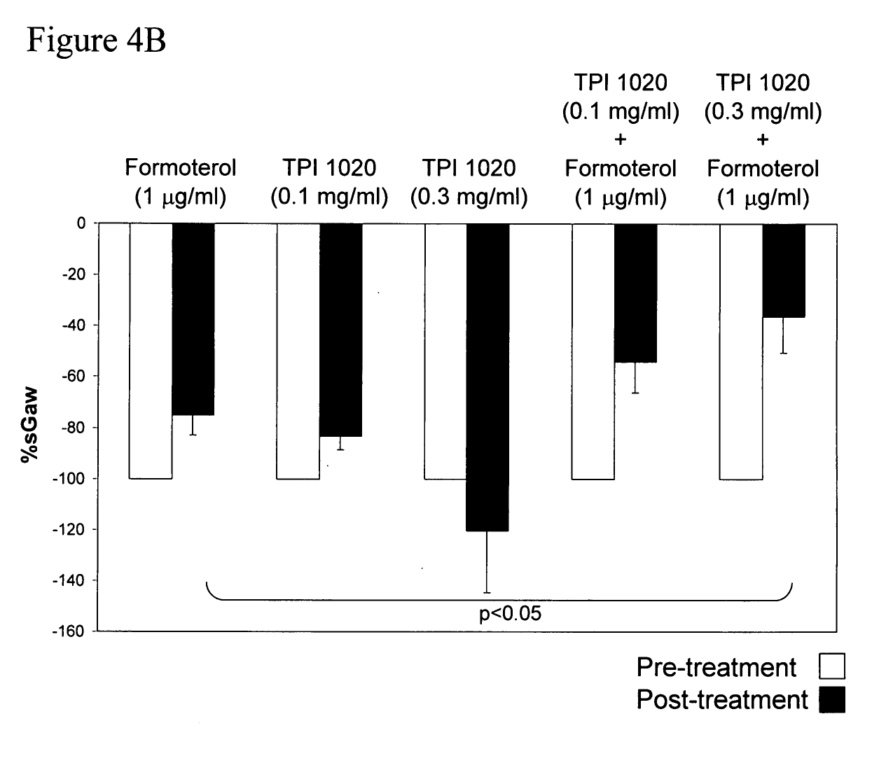

FIG. 4A shows the responsiveness of the airways to treatment with formoterol on bronchoconstriction induced by histamine. Baseline readings were taken on Day 1 prior to treatment (Pre-treatment) to determine 100% bronchoconstriction or AHR induced by histamine. On Day 2, animals were either treated with the vehicle or with 0.3, 1.0, or 3.0 ug/ml of formoterol 90 minutes before histamine challenge. Recordings of sG_{aw} were made at 0, 5 and 10 minutes after the histamine exposure (Post-treatment). sG_{aw} values of Day 2 are expressed as a percentage of Day 1 sG_{aw} values set at 100%. Each point represents the mean+/−S.E.M. (n=6). Statistical significance was determined for p<0.05 using One way ANOVA, post-hoc Tukeys.

Although each dose of formoterol showed significant reduction in AHR upon histamine challenge, both 0.3 and 1.0 ug/ml doses reduced AHR by less than 50%. A 0.3 mg/ml dose of TPI-1020 combined with a 1.0 ug/ml formoterol significantly reduced AHR by 60% (FIG. 4B), suggesting that, when used in combination with TPI-1020, efficacy can be achieved using a lower dose of bronchodilator, such as formoterol.

The results of the experiments presented in FIGS. 4A and 4B are presented in Table 4, below.

TABLE 4

| | Pre- | Post- | | |
|---|---|---|---|---|
| | treatment | treatment | | % |
| Treatment/Dose | (% sGaw) | (% sGaw) | SEM | Inhibition |
| FIG. 4A | | | | |
| Formoterol (μg/ml) | | | | |
| 0 | −100 | −100.4 | 13.2 | 0 |
| 0.3 | −100 | −70.0 | 14.4 | 30 |
| 1 | −100 | −75.1 | 7.9 | 25 |
| 3 | −100 | −29.2 | 16.6 | 71 |
| FIG. 4B | | | | |
| Formoterol (1 μg/ml) | −100 | −75.1 | 7.9 | 25 |
| TPI-1020 (0.1 mg/ml) | −100 | −83.2 | 5.6 | 17 |
| TPI-1020 (0.3 mg/ml) | −100 | −120.3 | 24.2 | −20 |
| TPI-1020 (0.1 mg/ml) + | −100 | −54.3 | 12.1 | 46 |
| Formoterol (1 μg/ml) | | | | |
| TPI-1020 (0.3 mg/ml) + | −100 | −36.2 | 14.2 | 64 |
| Formoterol (1 μg/ml) | | | | |

### Example 5

### Bronchodilating Effect of TPI-1020+Tiotropium

This example relates to experiments carried out to demonstrate that TPI-1020 potentiates the action of other classes of bronchodilators other than the beta-agonist class by showing the effects of the combination of TPI-1020 and tiotropium for the relief of bronchospasm induced by methacholine in guinea pigs.

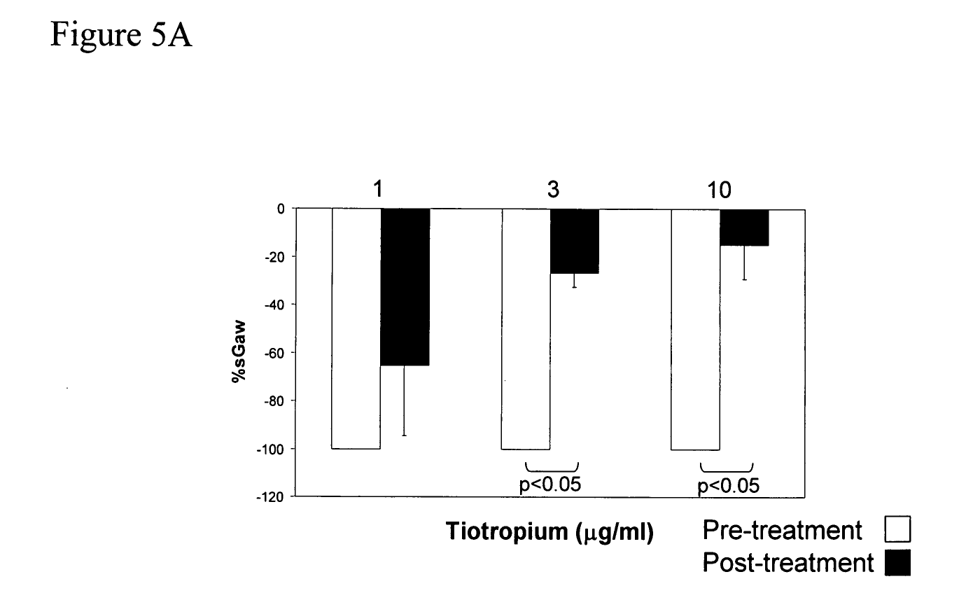

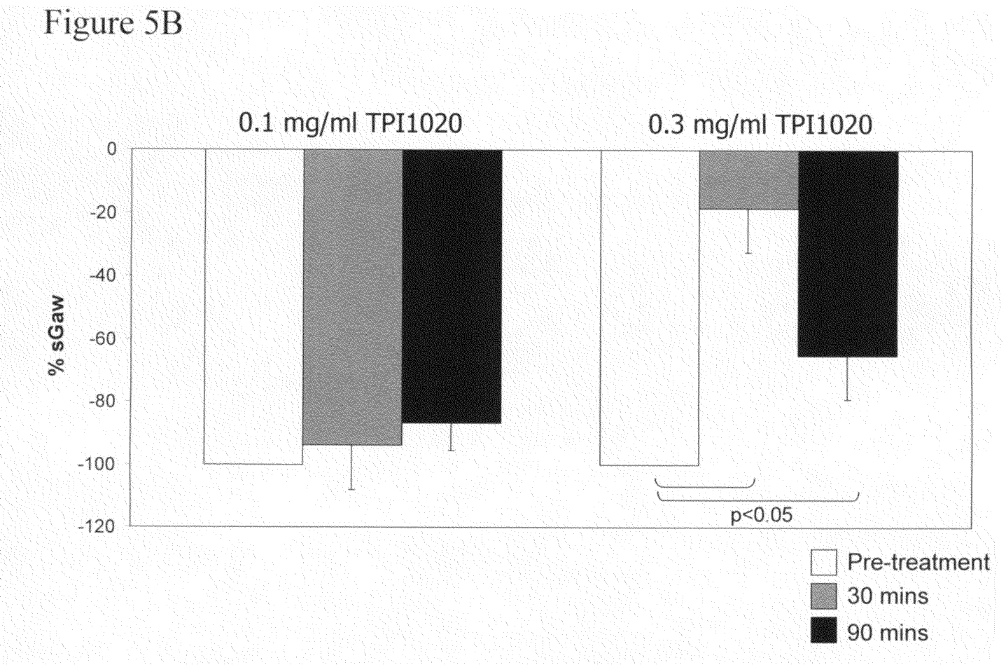

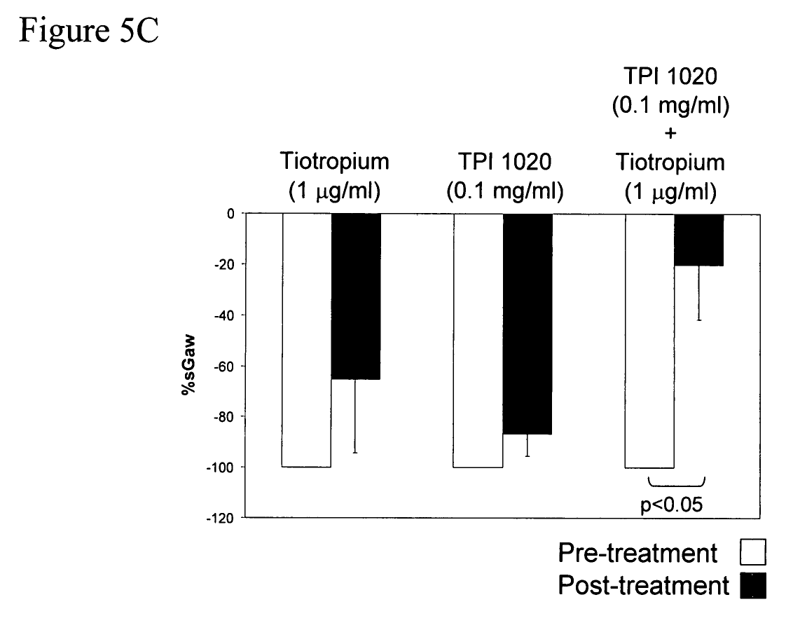

FIG. 5 shows the responsiveness of the airways to treatment with tiotropium on bronchoconstriction induced by methacholine. Tiotropium is an antimuscarinic or anticholinergic bronchodilator, different from the beta-agonist bronchodilators, such as salbutamol, described above. Baseline readings were taken on Day 1 prior to treatment (Pre-treatment) to determine 100% bronchoconstriction or AHR induced by methacholine. Recordings of sG_{aw} were made at 0, 5 and 10 minutes after the histamine exposure (Post-treatment). The sG_{aw} values of Day 3 are expressed as a percentage of Day 1 sG_{aw} values following methacholine challenge (Pre-treatment), which is by definition set at 100%. Each point represents the mean+/−S.E.M. (n=6). Statistical significance was determined for p<0.05 using Student's paired t-test.

On Day 3, animals were treated with vehicle, alone, or with 1.0, 3.0, or 10.0 ug/ml of tiotropium. FIG. 5A shows a significant reduction in AHR, versus pre-treatment, when using 3.0 and 10.0 ug/ml tiotropium (about 75% and 85% reduction of AHR, respectively). A dose of 0.1 mug/ml of tiotropium provided less than 40% reduction of AHR. FIG. 5B shows that, on Day 3, administration of 0.3 mg/ml TPI-1020 provides more than 80% reduction in AHR when administered 30 minutes before methacholine challenge, but less than 40% reduction in AHR when administered 90 minutes before methacholine challenge, as compared to AHR measured pre-treatment. Administration of 0.1 mg/ml of TPI-1020 provides less than 75% reduction in AHR as compared to Pre-treatment, when given 30 minutes or 90 minutes before methacholine challenge. However, as shown in FIG. 5C, TPI-1020 at a dose as small as 0.1 mg/ml in combination with a 1.0 ug/ml dose of tiotropium showed significant reduction in AHR. Specifically, FIG. 5C shows the combination of 0.1 mg/ml of TPI-1020 and 1.0 tiotropium reduced AHR 80%, whereas 1.0 ug/ml tiotropium, alone, reduced AHR less than 40% and 0.1 mg/ml TPI-1020, alone, reduced AHR less than 20%.

The results of the experiments presented in FIGS. 5A through 5C are presented in Table 5, below.

TABLE 5

| | Pre- | Post- | | |
|---|---|---|---|---|
| | treatment (% | treatment (% | | % |
| Treatment/Dose | sGaw) | sGaw) | SEM | Inhibition |
| FIG. 5A | | | | |
| Tiotropium (μg/ml) | | | | |
| 1 | −100 | −65.2 | 29.2 | 35 |
| 3 | −100 | −26.7 | 6.0 | 73 |
| 10 | −100 | −14.9 | 14.3 | 85 |
| FIG. 5B | | | | |
| TPI-1020 (0.1 mg/ml) | | | | |
| 30 mins | −100 | −94.0 | 13.9 | 6 |
| 90 mins | −100 | −86.9 | 8.7 | 13 |
| TPI-1020 (0.3 mg/ml) | | | | |
| 30 mins | −100 | −18.4 | 19.8 | 82 |
| 90 mins | −100 | −65.3 | 13.8 | 35 |
| FIG. 5C | | | | |
| Tiotropium (1 μg/ml) | −100 | −65.2 | 29.2 | 35 |
| TPI-1020 (0.1 mg/ml) | −100 | −86.9 | 8.7 | 13 |
| TPI-1020 (0.1 mg/ml) + | −100 | −20.3 | 21.4 | 80 |
| Tiotropium (1 μg/ml) | | | | |

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A pharmaceutical composition for treating a respiratory disease associated with inflammation in human patients, comprising
(i) at least one corticosteroid useful in the treatment of a respiratory disease; and
(ii) at least one bronchodilator selected from the group consisting of a beta-agonist, an anticholinergic, a methylxanthine, and a Phosphodiesterase inhibitor, or a physiologically acceptable salt or solvate thereof; and
(iii) a NO-donor wherein the donor is selected from the group consisting of an inactive carrier, a linking group linking the NO— to the corticosteroid, and a linking group linking the NO— to the bronchodilator.

2. The pharmaceutical composition of claim 1, wherein the NO-donor is linked to the corticosteroid.

3. The pharmaceutical composition of claim 1, wherein the NO-donor is linked to the bronchodilator.

4. The pharmaceutical composition of claim 1, wherein the NO-donor is a separate compound from the corticosteroid and the bronchodilator.

5. The pharmaceutical composition of claim 1, which is administrable via inhalation.

6. The pharmaceutical composition of claim 5, wherein the corticosteroid is in an amount suitable for treatment of a respiratory disease via inhalation in humans.

7. The pharmaceutical composition of claim 6, wherein the bronchodilator is in an amount suitable to provide an additional therapeutic effect.

8. The pharmaceutical composition of claim 7, wherein the combination of corticosteroid, bronchodilator, and NO-donor provide a synergistic effect in a human patient as compared to treatment of the patient without one of the agents.

9. The pharmaceutical composition of claim 1 wherein at least one of said corticosteroid, bronchodilator, or NO-donor is provided at a dose which is suboptimal if administered alone, and provides a significant reduction in bronchoconstriction or airway hyperreaction (AHR) following exposure to a bronchoconstriction-inducing agent, as compared to a composition substantially free of at least one of said corticosteroid, active agent, or NO-donor.

10. The pharmaceutical composition of claim 9 wherein the bronchoconstriction-inducing agent is histamine.

11. The pharmaceutical composition of claim 9 wherein the bronchoconstriction-inducing agent is methacholine.

12. The pharmaceutical composition of claim 9 wherein the significant reduction in bronchoconstriction is statistically significant to p<0.05.

13. The pharmaceutical composition of claim 9 wherein the significant reduction in bronchoconstriction is statistically significant to p<0.01.

14. The pharmaceutical composition of claim 1 wherein the composition provides a reduction in bronchoconstriction or airway hyperreaction (AHR) of at least 50%, following exposure to a bronchoconstriction-inducing agent, as compared to a composition free of at least one of said corticosteroid, active agent, or NO-donor.

15. The pharmaceutical composition of claim 14 wherein the reduction of bronchoconstriction or airway hyperreaction (AHR) is between about 70% and about 100%.

16. The pharmaceutical composition of claim 14 wherein at least one of said corticosteroid, bronchodilator, or NO-donor is provided at a dose which is suboptimal if administered alone.

17. The pharmaceutical composition of claim 8, wherein the dose of corticosteroid would be suboptimal if administered alone.

18. The pharmaceutical composition of claim 8, wherein the dose of the bronchodilator would be suboptimal if administered alone.

19. The pharmaceutical composition of claim 1, wherein the corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

20. The pharmaceutical composition of claim 1, wherein the formulation further comprises an additional active agent selected from the group consisting of a neurokinin receptor antagonist, a Leukotriene receptor antagonist, an antihistamine, an anti-inflammatory agent, a mast cell stabilizer, a CD137 agonist, a cytokine antagonist that antagonizes a cytokine selected from the group consisting of IL-4, IL-5, IL-9, IL-13 and/or combinations thereof, an Anti-IgE antibody, a 5-lipoxygenase inhibitor, a Monoamine Oxidase Inhibitor, a synthetic tryptamine, a mucolytic, a thromboxane receptor antagonist, and combinations of any of the foregoing.

21. The pharmaceutical composition of claim 11, wherein the NO-donor is linked to the corticosteroid to provide a compound of Formula (3):
A-W (3)
wherein A is the corticosteroid and wherein W is any nitric oxide (“NO”) donating moiety attached thereto capable of donating, releasing and/or directly or indirectly transferring any of the three redox forms of nitrogen monoxide (NO⁺, NO⁻, NO.).

22. The pharmaceutical composition of claim 1, wherein the NO-donor is linked to the corticosteroid to provide a compound of Formula (4): 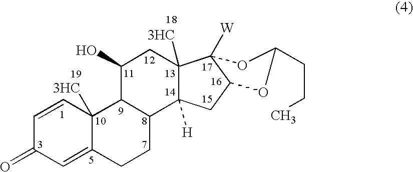
wherein W is any nitric oxide (“NO”) donating moiety attached thereto capable of donating, releasing and/or directly or indirectly transferring any of the three redox forms of nitrogen monoxide (NO⁺, NO⁻, NO.).

23. The pharmaceutical composition as claimed in claim 14, wherein W is selected from one of the Formulae A-G further wherein:
Formula A compounds being represented by: —C(O)-L-(X_{O})—(X₁)—NO₂ where L is defined as:
(CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;
X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and
X₁ is a bivalent-linking group selected from the following:
Y_{AR1} 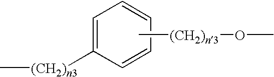
wherein n3 is an integer from 0 to 5 and n3′ is an integer from 1 to 3;
Y_{AR2} 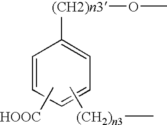
wherein n3 and n3′ have the above meaning, or
Y_{P} 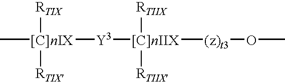
wherein:
nIX is an integer from 0 to 10, preferably 1-3;
nIIX is an integer from 1 to 10, preferably 1-5;
R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
t3 is zero or 1;
Z has the following meaning: 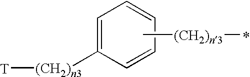
wherein:
shows the position of the ONO₂ group;
T has the following meanings:
—COX₃—, —X₃CO—, wherein X₃═S or X_{O} as above defined;
—X₃— as above defined;
n3 and n′3 are as above defined; 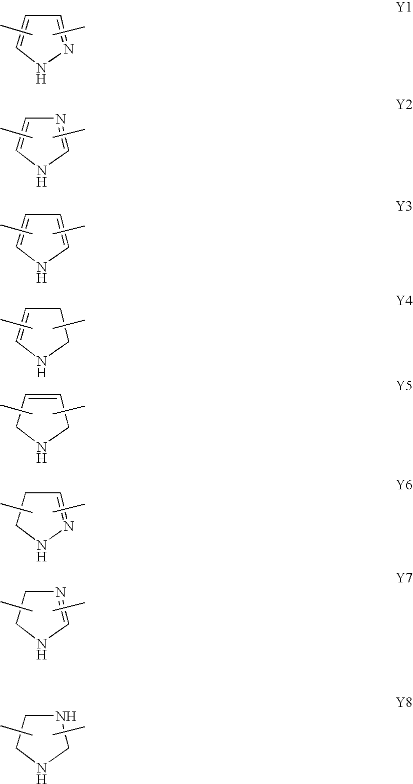
or Y³ is selected from the following bivalent radicals: 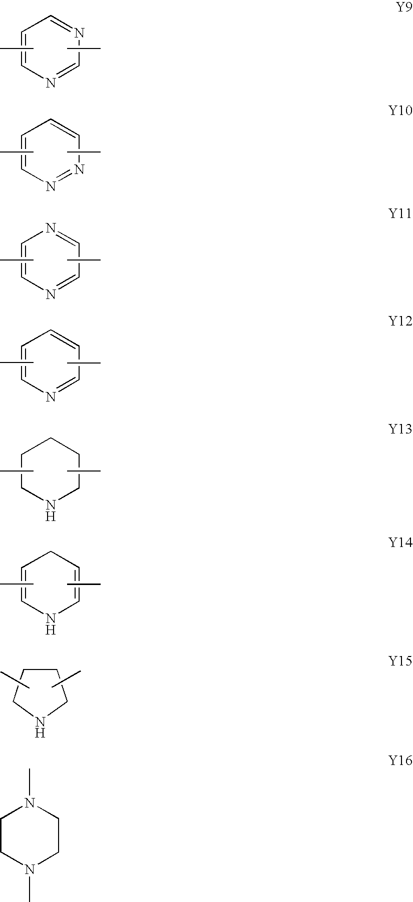
or is represented by:
—(CO-L)ₜ-(X₁)ₜ₁—X₁—NO₂
where t and t1 are integers=1 and L and X are defined as above as L and X₀, but where
X₁ is a bivalent-connecting bridge is selected from the group consisting of Y—O and Y₁, wherein:
for Y—O, Y is a linear or whenever possible branched C₁-C₂₀ alkylene, preferably having from 2 to 5 carbon atoms, or an optionally substituted cycloalkylene having from 5 to 7 carbon atoms; and
for Y₁, Y is selected from Y_{AR1, YAR2}, and Y_{P}, as defined above, and is more particularly: 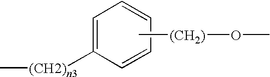
where n3 is an integer from 0 to 3; 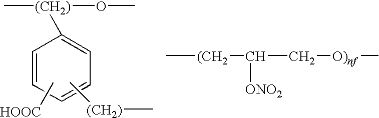
where nf′ is an integer from 1 to 6, preferably from 2 to 4; or 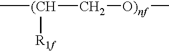
where R_{1f}═H, CH₃ and nf is an integer from 1 to 6, preferably from 2 to 4; or
is represented as:
—(CO-L)ₜ-(X)ₜ₁—X₁—NO₂
where t and t1 are integers=1 and L is defined as above;
wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;
X is equal to O, C═O, NH, NR_{1c} where R_{1c} is a C₁-C₁₀, and preferably a C₁-C₄ linear or branched alkyl; OH, CH₃, Cl, N(—CH₂—CH₃)₂, SCH₂F, SH, 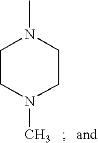
X₁ is a bivalent-connecting bridge is selected from the group consisting of Y—O and Y₁ as defined above; or
is represented as
C(O)-L-(X₀)—(X₁)—NO₂
wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, n″a, n′b, n″b and n′″b are 0 (thus L is CH₂O.)
X₀ is C═O, and
X₁ is the bivalent-linking group Y_{AR1}, preferably 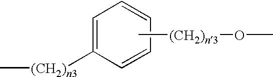
Formula B compounds being represented by: —C(O)CH₂O—X_{Z} or alternatively by —C(O)CH₂OC(O)—X_{Z} wherein X_{Z} is defined as follows: 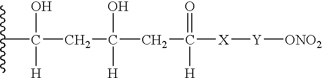
and X is O, S, NH or NHR₁, where R₁ is a straight or branched alkyl with 1 to 10 carbon atoms, preferably CH₃; and
Y is a bivalent radical having the following meanings a)-h)
a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl)-ONO₂ or is —(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃;
b) 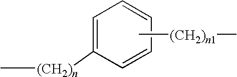
c) 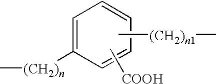
wherein for b) and c) above n is an integer from 0 to 20, and n¹ is an integer from 0 to 20;
d) 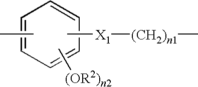
wherein, n¹ is as defined above and n² is an integer from 0 to 2; X₁ is —OCO— or —OCO— and R₂ is H or CH₃;
e) 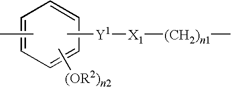
wherein n¹, n², R₂ and X₁ are as defined above; and Y¹ is either —CH₂—CH₂— or —CH₂═CH₂—(CH₂)ₙ₂—;
f) 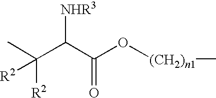
wherein n¹ and R² are as defined above; R³ is H or COCH₃; with the proviso that when Y is selected from the bivalent radicals mentioned under b) through f), the, —ONO₂ group is bound to —(CH₂)ₙ₁;
g) 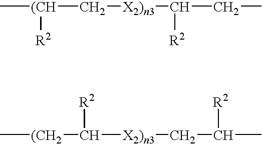
wherein X₂ is O or S, n³ is an integer from 1 to 6, preferably from 1 to 4, and R² is defined above;
h) 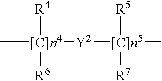
wherein: n⁴ is an integer from 0 to 10; n is an integer from 1 to 10; R⁴, R⁵, R⁶, and R⁷ are the same or different, and are H or straight or branched C₁-C₁₀ alkyl; and preferably R⁴, R⁵R⁶, and R⁷ are H; wherein the —ONO₂ group is bound to the following structure: 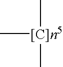
wherein n⁵ is defined above;
Y² is a heterocyclic saturated, unsaturated or aromatic 5 or 6 members ring, containing one or more heteroatoms selected from nitrogen, oxygen, sulfur, and is selected from the following structures H1 through H13: 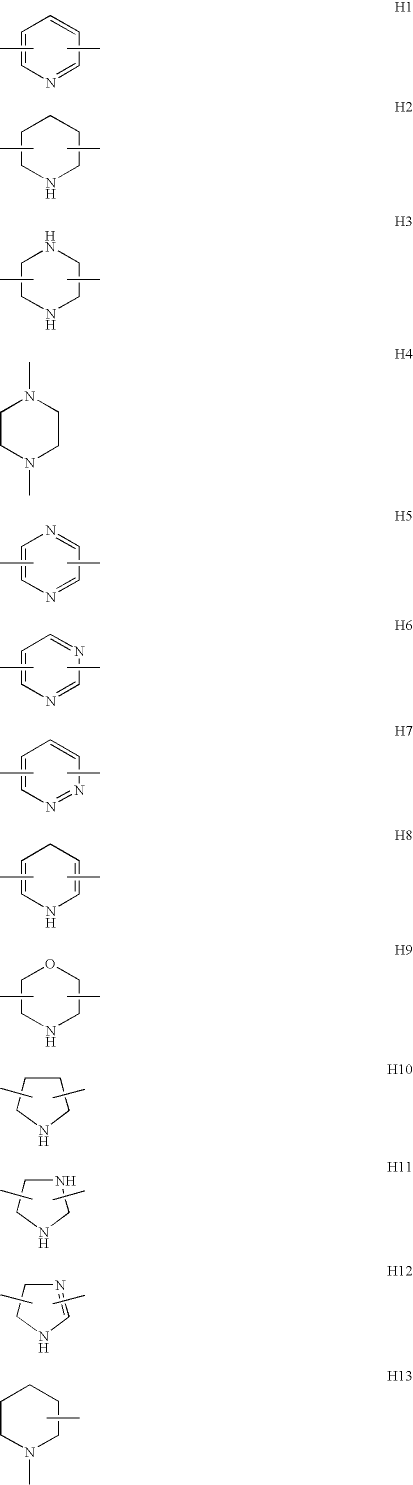
Formula C compounds being represented by: —C(O)CH₂O—(X₁—ONO₂)_{S} or alternatively by —C(O)CH₂OC(O)—(X₁—ONO₂)_{S} wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—;
Formula D compounds being represented by: —C(O)CH₂O—B—C) wherein X₁ is defined as follows: 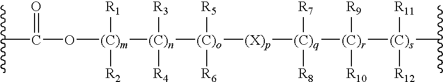
where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above;
Formula E compounds being represented by: —C(O)CH₂O—B—C^{Z}, where C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO donating portion of the compound, C^{Z}, via an amide, ester, carbamate or carbonate linkage that is induced adjacent to the 21 position;
wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
B—C^{Z} is equal to the following structure: 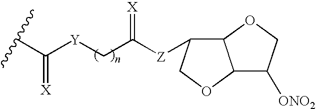
wherein n is an integer from 1 to 4; X═O or S; Y=methylene, O, or NH₂; and Z=O or NH₂; or where
W=Formula E′ where E′ is —(O)CH₂O—(B′—C^{Z}) wherein B′ is a spacer preferably containing 12 carbon atoms or less that connects the steroid backbone at the hydroxy immediately adjacent to the C-21 position with the NO donating portion of the compound, C^{Z}, via an amide, ester, carbamate or carbonate linkage that is not adjacent to the C-21 position; or
Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
R₁ can be selected from any of the following chemical moieties that have been appropriately substituted with a NO donating group: lower alkyls/alkenyls/alkynyls; that are substituted or unsubstituted; substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.
Formula F compounds being represented by: —C(O)CH₂O—K or alternatively by —C(O)CH₂OC(O)—K wherein K is defined as follows:
—Y—(CR₄R_{4′})ₚ-T-(CR₄R_{4′})ₚ—ONO₂;
—Y—(CR₄R_{4′})ₒ-[phenyl]-T-(CR₄R_{4′})ₚ—ONO₂;
wherein T is ortho, meta or para;
—Y—B-[piperazinyl]-W—(CR₄R_{4′})ₚ—ONO₂;
—Y—(CR₄R_{4′})ₚ—V—B-T-(CR₄R_{4′})ₚ—ONO₂;
—Y—(CR₄R_{4′})ₚ-T-C(O)—(CR₄R_{4′})ₚ—(CH₂)—ONO₂;
—Y—(CR₄R_{4′})ₚ—C(Z)-(CH₂)_{q}-T-(CR₄R_{4′})_{q}—(CH₂)—ONO₂;
—Y—(CR₄R_{4′})ₚ-T-(CH₂)_{q}—V—(CR₄R_{4′})_{q}—(CH₂)—ONO₂;
—Y—(CR₄R_{4′})ₚ—V—(CH₂)_{q}—V—(CR₄R_{4′})_{q}—(CH₂)—ONO₂;
—Y—(CR₄R_{4′})ₒ—(W)_{q}—(CH₂)_{q}—V—(CR₄R_{4′})ₒ—(CH₂)—ONO₂;
—NRⱼ—O—(CH₂)ₒ—V—(CR₄R_{4′})_{q}—(CH₂)—ONO₂;
—NRⱼ—O—(CH₂)ₒ—(W)_{q}—(CR₄R_{4′})_{q}—(CH₂)—ONO₂;
—O—NRⱼ—(CH₂)ₒ—(W)_{q}—(CR₄R_{4′})_{q}—(CH₂)—ONO₂;
Formula G compounds being represented by: —C(O)CH₂O—X1 or alternatively by —C(O)CH₂OC(O)—X1 wherein X1 is defined as follows:
—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;
or alternatively, X1 is equal to K which is further defined as:
—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);
where —(U)—(V) is equal to —Wᵢ-Ej-W_{g}—[C—(Rₑ)(R_{f})]_{z}-T-Q; or alternatively U and V are taken independently wherein U is O, S, or —N(Rₐ)(Rᵢ) and V is nitro, nitroso, or hydrogen;
wherein a, b, c, d, g, i and j are each independently an integer from 0 to 3;
p, x, y and z are each independently an integer from 0 to 10;
W is independently —C(O)—, —C(S)—, -T-, —[C—(Rₑ)(R_{f})]ₕ—, an alkyl group, an aryl group, a heterocyclic ring, an arylheterocyclic ring, or —(CH₂CH₂O)_{q}—;
E at each occurrence is independently -T-, an alkyl group, an aryl group, —[C—(Rₑ)(R_{f})]ₕ—, a heterocyclic ring, an arylheterocyclic ring, or —(CH₂CH₂O)_{q}—;
h is an integer form 1 to 10;
q is an integer of from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;
k is an integer from 1 to 3;
T at each occurrence is independently a covalent bond, a carbonyl, an oxygen, —S(O)ₒ— or —N(Rₐ)(Rᵢ)—;
o is an integer from 0 to 2;
Rₐ is a lone pair of electrons, a hydrogen or an alkyl group;
Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ-T-Q, then the “-T-Q” subgroup designated as X can be a hydrogen, an alkyl, an alkoxy, an alkoxyalkyl, an aminoalkyl, a hydroxy, a heterocyclic ring or an aryl group, wherein, in cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

24. The pharmaceutical composition of claim 1, wherein the corticosteroid and NO-donor have the structure of Formula 1: 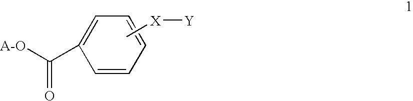
wherein A is a corticosteroid selected from the group consisting of 21-acetoxypregnenolone, alclometasone, algestone, amcinonide, beclomethasone, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide;
X is a C₁-C₅ branched or linear chain alkyl; and
Y is either (ONO₂) or (ONO); with the proviso that A is linked to the remainder of Formula 1 by way of the C-11 or the C-17 position.

25. The pharmaceutical composition as claimed in claim 15, wherein A is selected from budesonide, X is CH₂ and Y is (ONO₂) and A is linked to the remainder of Formula 1 through the C-17 position.

26. The pharmaceutical composition of claim 1, wherein the corticosteroid is budesonide and the NO-donor is linked to the budesonide to provide a compound of Formula 2:

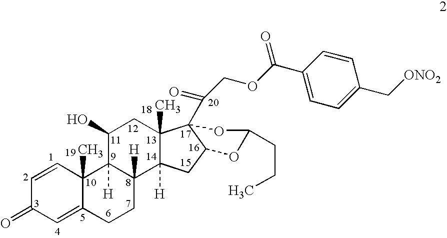

27. The pharmaceutical composition-of claim 1, wherein the bronchodilator is a β₂-agonist.

28. The pharmaceutical composition-of claim 1, wherein the at least one bronchodilator is an anticholinergic.

29. The pharmaceutical composition as claimed in claim 7, wherein the β₂-agonist is selected from the group consisting of salbutamol, bitolterol mesylate, formoterol, isoproterenol, levalbuterol, metaproterenol, salmeterol, terbutaline, and fenoterol.

30. The pharmaceutical composition as claimed in claim 27, wherein the β₂-agonist is a short-acting β₂-agonist.

31. The pharmaceutical composition as claimed in claim 30, wherein the short-acting β₂-agonist is salbutamol.

32. The pharmaceutical composition as claimed in claim 31, wherein the weight ratio of the compound of Formula 1 to salbutamol is from about 1:8 to about 4:1.

33. The pharmaceutical composition as claimed in claim 27, wherein the β₂-agonist is a long-acting β₂-agonist.

34. The pharmaceutical composition as claimed in claim 33, wherein the long-acting β₂-agonist is formoterol or a physiologically acceptable salt or solvate thereof.

35. The pharmaceutical composition as claimed in claim 28, wherein the anticholinergic is selected from tiotropium or ipratropium.

36. The pharmaceutical composition of claim 1, wherein the at least one bronchodilator comprises a β₂-agonist and an anticholinergic.

37. The pharmaceutical composition of claim 1, wherein the respiratory disease is a chronic pulmonary disease.

38. The pharmaceutical composition as claimed in claim 37, wherein the chronic pulmonary disease is selected from asthma and COPD.

39. The pharmaceutical composition of claim 1, wherein the respiratory disease is selected from the group consisting of eosinophilic cough, bronchitis, sarcoidosis, pulmonary fibrosis, pulmonary hypertension, cystic fibrosis, and bronchiectasis.

40. The pharmaceutical composition of claim 1, wherein the NO-donor is S-nitroso-N-acetylpenicillamine (SNAP).

41. The pharmaceutical composition of claim 40, wherein the corticosteroid is budesonide.

42. A method for treating a respiratory disease associated with inflammation, said method comprising simultaneously or sequentially administering to a patient in need thereof a therapeutically effective amount of (i) at least one corticosteroid useful in the treatment of, simultaneously or sequentially administered with (ii) at least one bronchodilator selected from the group consisting of a beta-agonist, an anticholinergic, a methylxanthine, and a Phosphodiesterase inhibitor, or a physiologically acceptable salt or solvate thereof; and (iii) an NO-donor wherein the donor is selected from the group consisting of an inactive carrier, a linking group linking the NO— to the corticosteroid, and a linking group linking the NO— to the bronchodilator.

43. The method of claim 42, wherein the NO-donor is linked to the corticosteroid.

44. The method of claim 42, wherein the NO-donor is linked to the bronchodilator.

45. The method of claim 42, wherein the NO-donor is a separate compound from the corticosteroid and the active agent.

46. The method of claim 42, which is administrable via inhalation.

47. The method of claim 44, wherein the corticosteroid is in an amount suitable for treatment of a respiratory disease via inhalation in humans.

48. The method of claim 42, wherein the bronchodilator is in an amount suitable to provide an additional therapeutic effect.

49. The method of claim 42, wherein the combination of corticosteroid, active agent, and NO-donor provide a synergistic effect in a human patient as compared to treatment of the patient without one of the agents.

50. The method of claim 49, wherein the dose of corticosteroid would be suboptimal if administered alone.

51. The method of claim 41, wherein the dose of the active agent would be suboptimal if administered alone.

52. The method of claim 41 wherein at least one of said corticosteroid, bronchodilator, or NO-donor is provided at a dose which is suboptimal if administered alone, and provides a significant reduction in bronchoconstriction or airway hyperreaction (AHR) following exposure to a bronchoconstriction-inducing agent, as compared to a composition substantially free of at least one of said corticosteroid, active agent, or NO-donor.

53. The method of claim 52 wherein the bronchoconstriction-inducing agent is histamine.

54. The method of claim 52 wherein the bronchoconstriction-inducing agent is methacholine.

55. The method of claim 52 wherein the significant reduction in bronchoconstriction is statistically significant to p<0.05.

56. The method of claim 52 wherein the significant reduction in bronchoconstriction is statistically significant to p<0.01.

57. The method of claim 41 wherein the composition provides a reduction in bronchoconstriction or airway hyperreaction (AHR) of at least 50%, following exposure to a bronchoconstriction-inducing agent, as compared to a composition free of at least one of said corticosteroid, active agent, or NO-donor.

58. The method of claim 57 wherein the reduction of bronchoconstriction or airway hyperreaction (AHR) is between about 70% and about 100%.

59. The method of claim 57 wherein at least one of said corticosteroid, bronchodilator, or NO-donor is provided at a dose which is suboptimal if administered alone.

60. The method of claim 42, wherein the corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

61. The method of claim 42, further comprising the simultaneous or sequential administration of an additional active agent selected from the group consisting of a neurokinin receptor antagonist, a Leukotriene receptor antagonist, an antihistamine, an anti-inflammatory agent, a mast cell stabilizer, a CD137 agonist, a cytokine antagonist that antagonizes a cytokine selected from the group consisting of IL-4, IL-5, IL-9, IL-13, and/or combinations thereof, an Anti-IgE antibody, a 5-lipoxygenase inhibitor, a Monoamine Oxidase Inhibitor, a synthetic tryptamine, a mucolytic, a thromboxane receptor antagonist, and combinations of any of the foregoing.

62. The method of claim 42, wherein the respiratory disease is a chronic pulmonary disease.

63. The method of claim 62, wherein the chronic pulmonary disease is selected from asthma and COPD.

64. The method of claim 42, wherein the respiratory disease is selected from the group consisting of eosinophilic cough, bronchitis, sarcoidosis, pulmonary fibrosis, pulmonary hypertension, cystic fibrosis, and bronchiectasis.

65. The method of claim 42, wherein the at least one bronchodilator is selected from a β₂-agonist and an anticholinergic.

66. The method of claim 65, wherein the bronchodilator is a β₂-agonist.

67. The method of claim 66, wherein the β₂-agonist is a short-acting β₂-agonist.

68. The method of claim 67, wherein the short-acting β₂-agonist is salbutamol, or a physiologically acceptable sat or solvate thereof.

69. The method of claim 68, wherein the corticosteroid is administered in a weight ratio relative to salbutamol, or a physiologically acceptable salt or solvate thereof, of from about 1:8 to about 4:1.

70. The method of claim 66, wherein the β₂-agonist is a long-acting β₂-agonist.

71. The method of claim 70, wherein the long-acting β₂-agonist is formoterol or a physiologically acceptable salt or solvate thereof.

72. The method of claim 42, wherein the bronchodilator is an anticholinergic.

73. The method of claim 72, wherein the anticholinergic is selected from tiotropium or ipratropium.

74. The method as claimed in claim 50, wherein the bronchodilator is administered orally.

75. The method as claimed in claim 50, wherein the bronchodilator and the compound of Formula 1 are administered by inhalation.

76. The method as claimed in claim 50, wherein the NO-donor is linked to the corticosteroid to provide a compound of Formula (4): 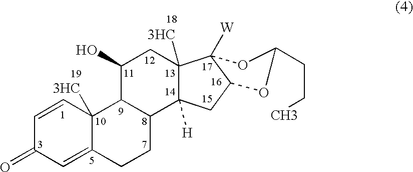
wherein W is any nitric oxide (“NO”) donating moiety attached thereto capable of donating, releasing and/or directly or indirectly transferring any of the three redox forms of nitrogen monoxide (NO⁺, NO⁻, NO.).

77. The method as in claim 76 wherein W is selected from one of the Formulae A-G further wherein:
Formula A compounds being represented by: —C(O)-L-(X_{O})—(X₁)—NO₂ where L is defined as:
(CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;
X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and
X₁ is a bivalent-linking group selected from the following:
Y_{AR1} 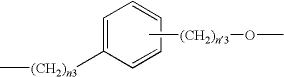
wherein n3 is an integer from 0 to 5 and n3′ is an integer from 1 to 3;
Y_{AR2} 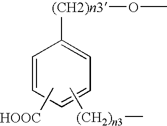
wherein n3 and n3′ have the above meaning, or
Y_{P} 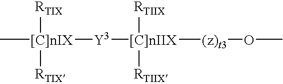
wherein:
nIX is an integer from 0 to 10, preferably 1-3;
nIIX is an integer from 1 to 10, preferably 1-5;
R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
t3 is zero or 1;
Z has the following meaning: 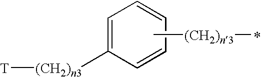
wherein:
shows the position of the ONO₂ group;
T has the following meanings:
—COX₃—, —X₃CO—, wherein X₃═S or X_{O} as above defined;
—X₃— as above defined;
n3 and n′3 are as above defined;
or Y³ is selected from the following bivalent radicals: 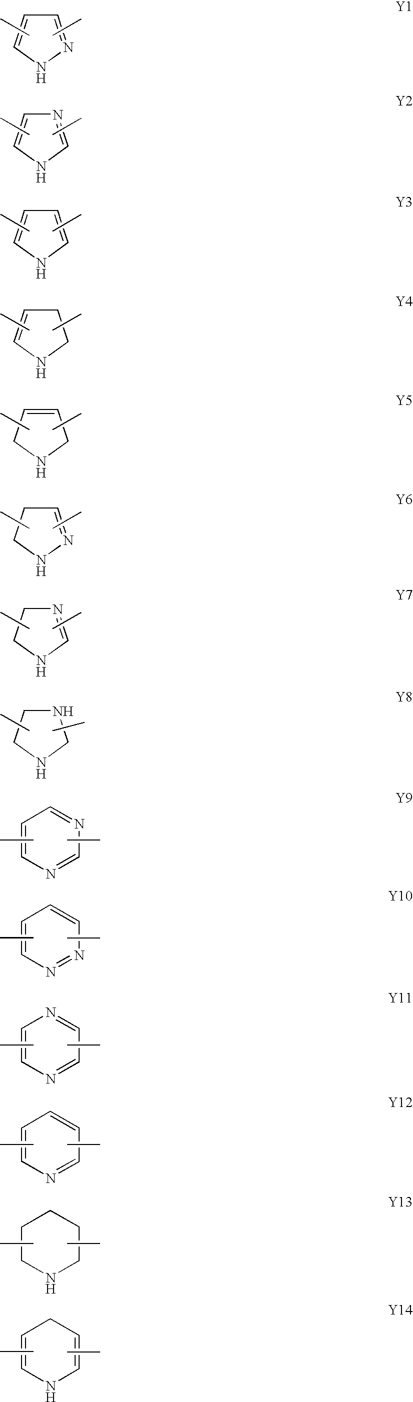 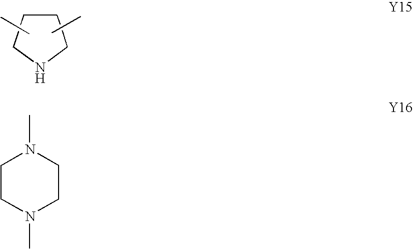
or is represented by:
—(CO-L)ₜ-(X)ₜ₁—X₁—NO₂
where t and t1 are integers=1 and L and X are defined as above as L and X₀, but where
X₁ is a bivalent-connecting bridge is selected from the group consisting of Y—O and Y₁, wherein:
for Y—O, Y is a linear or whenever possible branched C₁-C₂₀ alkylene, preferably having from 2 to 5 carbon atoms, or an optionally substituted cycloalkylene having from 5 to 7 carbon atoms; and
for Y₁, Y is selected from Y_{AR1, YAR2}, and Y_{P}, as defined above, and is more particularly: 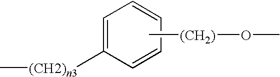
where n3 is an integer from 0 to 3; 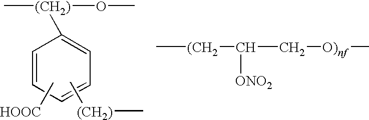
where nf′ is an integer from 1 to 6, preferably from 2 to 4; or 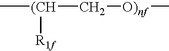
where R_{1f}═H, CH₃ and nf is an integer from 1 to 6, preferably from 2 to 4; or
is represented as:
—(CO-L)ₜ-(X)ₜ₁—X₁—NO₂
where t and t1 are integers=1 and L is defined as above:
wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;
X is equal to O, C═O, NH, NR_{1c} where R_{1c} is a C₁-C₁₀, and preferably a C₁-C₄ linear or branched alkyl; OH, CH₃, Cl, N(—CH₂—CH₃)₂, SCH₂F, SH, 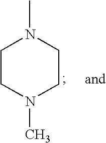
X₁ is a bivalent-connecting bridge is selected from the group consisting of Y—O and Y₁ as defined above; or
is represented as
C(O)-L-(X₀)—(X₁)—NO₂
wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, n″a, n′b, n″b and n′″b are 0 (thus L is CH₂O.)
X₀ is C═O, and
X₁ is the bivalent-linking group Y_{AR1}, preferably 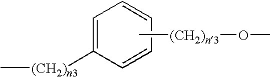
Formula B compounds being represented by: —C(O)CH₂O—X_{Z} or alternatively by —C(O)CH₂OC(O)—X_{Z} wherein X_{Z} is defined as follows: 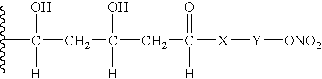
and X is O, S, NH or NHR₁, where R₁ is a straight or branched alkyl with 1 to 10 carbon atoms, preferably CH₃; and
Y is a bivalent radical having the following meanings a)-h)
a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl)-ONO₂ or is —O(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃;
b) 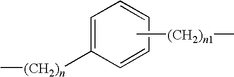
c) 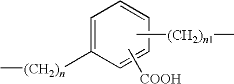
wherein for b) and c) above n is an integer from 0 to 20, and n¹ is an integer from 0 to 20;
d) 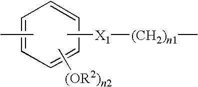
wherein, n¹ is as defined above and n² is an integer from 0 to 2; X₁ is —OCO— or —OCO— and R₂ is H or CH₃;
e) 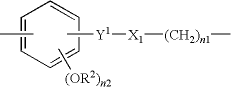
wherein n¹, n², R₂ and X₁ are as defined above; and Y¹ is either —CH₂—CH₂— or —CH₂═CH₂—(CH₂)ₙ₂—;
f) 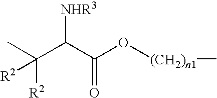
wherein n¹ and R² are as defined above; R³ is H or COCH₃; with the proviso that when Y is selected from the bivalent radicals mentioned under b) through f), the, —ONO₂ group is bound to —(CH₂)ₙ₁;
g) 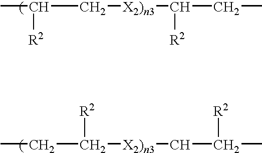
wherein X₂ is O or S, n³ is an integer from 1 to 6, preferably from 1 to 4, and R² is defined above;
h) 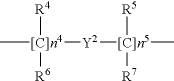
wherein: n⁴ is an integer from 0 to 10; n⁵ is an integer from 1 to 10; R⁴, R⁵, R⁶, and R⁷ are the same or different, and are H or straight or branched C₁-C₁₀ alkyl; and preferably R⁴, R⁵R⁶, and R⁷ are H; wherein the —ONO₂ group is bound to the following structure: 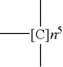
wherein n⁵ is defined above;
Y² is an heterocyclic saturated, unsaturated or aromatic 5 or 6 members ring, containing one or more heteroatoms selected from nitrogen, oxygen, sulfur, and is selected from the following structures H1 through H13: 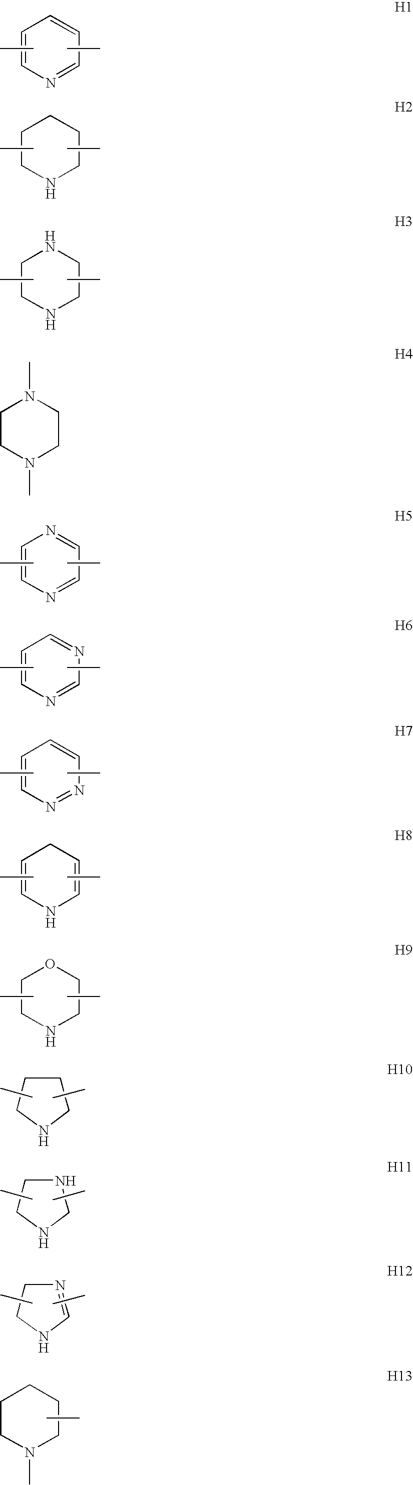
Formula C compounds being represented by: —C(O)CH₂O—(X₁—ONO₂)_{S} or alternatively by —C(O)CH₂OC(O)—(X₁—ONO₂)_{S} wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—;
Formula D compounds being represented by: —(O)CH₂O—B—C) wherein X₁ is defined as follows: 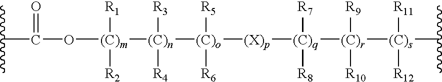
where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above;
Formula E compounds being represented by: —C(O)CH₂O—B—C^{Z}, where C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO donating portion of the compound, C^{Z}, via an amide, ester, carbamate or carbonate linkage that is induced adjacent to the 21 position;
wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
B—C^{Z} is equal to the following structure: 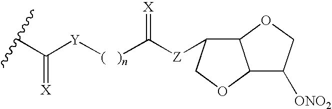
wherein n is an integer from 1 to 4; X═O or S; Y=methylene, O, or NH₂; and Z=O or NH₂; or where
W=Formula E′ where E′ is —C(O)CH₂O—(B′—C^{Z}) wherein B′ is a spacer preferably containing 12 carbon atoms or less that connects the steroid backbone at the hydroxy immediately adjacent to the C-21 position with the NO donating portion of the compound, C^{Z}, via an amide, ester, carbamate or carbonate linkage that is not adjacent to the C-21 position; or
Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
R₁ can be selected from any of the following chemical moieties that have been appropriately substituted with a NO donating group: lower alkyls/alkenyls/alkynyls; that are substituted or unsubstituted; substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.
Formula F compounds being represented by: —(O)CH₂O—K or alternatively by —C(O)CH₂OC(O)—K wherein K is defined as follows:
—Y—(CR₄R_{4′})ₚ-T-(CR₄R_{4′})ₚ—ONO₂;
—Y—(CR₄R_{4′})ₒ-[phenyl]-T-(CR₄R_{4′})ₚ—ONO₂;
wherein T is ortho, meta or para;
—Y—B-[piperazinyl]-W—(CR₄R_{4′})ₚ—ONO₂;
—Y—(CR₄R_{4′})ₚ—V—B-T-(CR₄R_{4′})ₚ—ONO₂;
—Y—(CR₄R_{4′})ₚ-T-C(O)—(CR₄R_{4′})ₚ—(CH₂)—ONO₂;
—Y—(CR₄R_{4′})ₚ—C(Z)-(CH₂)_{q}-T-(CR₄R_{4′})_{q}—(CH₂)—ONO₂;
—Y—(CR₄R_{4′})ₚ-T-(CH₂)_{q}—V—(CR₄R_{4′})_{q}—(CH₂)—ONO₂;
—Y—(CR₄R_{4′})ₚ—V—(CH₂)_{q}—V—(CR₄R_{4′})_{q}—(CH₂)—ONO₂;
—Y—(CR₄R_{4′})ₒ—(W)_{q}—(CH₂)_{q}—V—(CR₄R_{4′})ₒ—(CH₂)—ONO₂;
—NRⱼ—O—(CH₂)ₒ—V—(CR₄R_{4′})_{q}—(CH₂)—ONO₂;
—NRⱼ—O—(CH₂)ₒ—(W)_{q}—(CR₄R_{4′})_{q}—(CH₂)—ONO₂;
—O—NRⱼ—(CH₂)ₒ—(W)_{q}—(CR₄R_{4′})_{q}—(CH₂)—ONO₂;
Formula G compounds being represented by: —C(O)CH₂O—X1 or alternatively by —C(O)CH₂OC(O)—X1 wherein X1 is defined as follows:
—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;
or alternatively, X1 is equal to K which is further defined as:
—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);
where —(U)—(V) is equal to —Wᵢ-Ej-W_{g}—[C—(Rₑ)(R_{f})]_{z}-T-Q; or alternatively U and V are taken independently wherein U is O, S, or —N(Rₐ)(Rᵢ) and V is nitro, nitroso, or hydrogen;
wherein a, b, c, d, g, i and j are each independently an integer from 0 to 3;
p, x, y and z are each independently an integer from 0 to 10;
W is independently —(O)—, —C(S)—, -T-, —[—C—(Rₑ)(R_{f})]ₕ—, an alkyl group, an aryl group, a heterocyclic ring, an arylheterocyclic ring, or —(CH₂CH₂O)_{q}—;
E at each occurrence is independently -T-, an alkyl group, an aryl group, —[C—(Rₑ)(R_{f})]ₕ—, a heterocyclic ring, an arylheterocyclic ring, or —(CH₂CH₂O)_{q}—;
h is an integer form 1 to 10;
q is an integer of from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;
k is an integer from 1 to 3;
T at each occurrence is independently a covalent bond, a carbonyl, an oxygen, —S(O)ₒ— or —N(Rₐ)(Rᵢ)—;
o is an integer from 0 to 2;
Rₐ is a lone pair of electrons, a hydrogen or an alkyl group;
Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ-T-Q, then the “-T-Q” subgroup designated as X can be a hydrogen, an alkyl, an alkoxy, an alkoxyalkyl, an aminoalkyl, a hydroxy, a heterocyclic ring or an aryl group, wherein, in cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

78. The method as claimed in claim 50, wherein the corticosteroid and NO-donor have the structure of Formula 1: 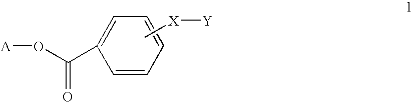
wherein A is a corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide;
X is a C₁-C₅ branched or linear chain alkyl; and
Y is either (ONO₂) or (ONO); with the proviso that A is linked to the remainder of Formula 1 by way of the C-11 or the C-17 position.

79. The method of claim 78, wherein A is selected from budesonide, X is CH₂ and Y is (ONO₂) and A is linked to the remainder of Formula 1 through the C-17 position.

80. The method as claimed in claim 50, wherein the corticosteroid is budesonide and the NO-donor is linked to the budesonide to provide a compound of Formula 2:

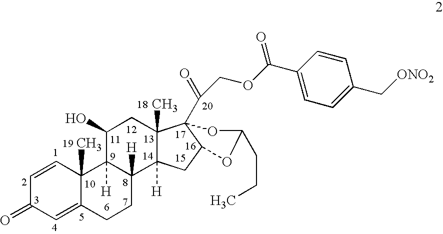

81. A method for treating respiratory disease associated with inflammation in a patient in need thereof, said method comprising simultaneously or sequentially administering a therapeutically effective amount of (i) at least one bronchodilator, or a physiologically acceptable salt or solvate thereof, and (ii) a compound of Formula 2: [chemical structure]
